(19) United States
(12) Patent Application Publication
(10) Pub. No.: US 2024/0374396 A1
(43) Pub. Date: Nov. 14, 2024

(54) INTERBODY FUSION DEVICE AND METHODS OF USE THEREOF

(71) Applicant: Board of Supervisors of Louisiana State University and Agricultural and Mechanical College, Baton Rouge, LA (US)

(72) Inventors: Giovanni SOLITRO, Shreveport, LA (US); Milan G. MODY, Shreveport, LA (US); R Shane BARTON, Shreveport, LA (US)

(21) Appl. No.: 18/661,492

(22) Filed: May 10, 2024

Related U.S. Application Data
(60) Provisional application No. 63/465,359, filed on May 10, 2023.

(51) Int. Cl. A61F 2/44; A61F 2/30

(52) U.S. Cl. CPC A61F 2/4455; A61F 2002/30433; A61F 2002/30538; A61F 2002/3055; A61F 2002/30579; A61F 2002/448

(57) ABSTRACT

A device is described herein comprising lateral bodies configured for contact with a vertebral endplate of a subject, wherein the lateral bodies comprise a first lateral body and a second lateral body, wherein the first lateral body and the second lateral body are connected using linking elements, wherein the linking elements comprise a first linking element and a second linking element; wherein the linking elements are configured for adjustable spacing of the lateral bodies relative to each other.

All patents, patent applications, and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### RELATED APPLICATIONS

This application claims priority to U.S. Application No. 63/465,359, filed May 10, 2023, and which is incorporated herein by reference in its entirety.

### FIELD OF THE INVENTION

This invention is directed to interbody fusion devices and kits, and methods for using the same.

### BACKGROUND OF THE INVENTION

For the treatment of multiples spinal pathologies vertebral interbody fusion can be used. Failure of interbody spacers can be documented in the form of subsidence, for example in patients with osteoporosis, for which interbody fusion is can be contraindicated.

### SUMMARY OF THE INVENTION

In embodiments, a device is described comprising lateral bodies configured for contact with a vertebral endplate of a subject, wherein the lateral bodies comprise a first lateral body and a second lateral body, wherein the first lateral body and the second lateral body are connected using linking elements, wherein the linking elements comprise a first linking element and a second linking element, wherein the linking elements are configured for adjustable spacing of the lateral bodies relative to each other.

In embodiments, the adjustable spacing comprises deformation of at least one of the first linking element and the second linking element.

In embodiments, the deformation comprises expansion or contraction of the at least one of the first linking element and the second linking element.

In embodiments, the first linking element connects corresponding posterior portions of the lateral bodies.

In embodiments, expansion of the first linking element comprises expanding distance between the corresponding posterior portions.

In embodiments, compression of the first linking element comprises contraction of distance between the corresponding posterior portions.

In embodiments, the second linking element connects corresponding anterior portions of the lateral bodies.

In embodiments, expansion of the second linking element comprises expanding distance between the corresponding anterior portions.

In embodiments, compression of the second linking element comprises contraction of distance between the corresponding anterior portions.

In embodiments, the lateral bodies and the linking elements form an interior space.

In embodiments, at least one of the first and the second lateral body comprises a lateral body interior space.

In embodiments, a device comprises lateral bodies configured for contact with a vertebral endplate of a subject, wherein the lateral bodies comprise a first lateral body and a second lateral body, wherein the first lateral body and the second lateral body are connected using linking elements, wherein the linking elements comprise a first linking element and a second linking coupling, wherein the linking elements are configured for adjustable spacing of the lateral bodies relative to each other.

In embodiments, the first linking element connects corresponding posterior portions of the first lateral body and the second lateral body, wherein the first linking element is deformable.

In embodiments, the linking coupling connects corresponding anterior portions of the second lateral body.

In embodiments, the linking coupling comprises a sliding component integrally formed with and extending from the first lateral body.

In embodiments, the linking coupling comprises a receiving chamber formed within the second lateral body, wherein the receiving chamber comprises an enclosed distal end and a proximal opening.

In embodiments, movement of the sliding component in a direction of the closed distal end of the receiving chamber corresponds to compression of the first linking element and contraction of distance between the lateral bodies.

In embodiments, movement of the sliding component in a direction away from the closed distal end of the receiving chamber corresponds to expansion of the first linking element and expansion of distance between the lateral bodies.

In embodiments, the sliding component and the receiving chamber are linear shaped and the movement is linear.

In embodiments, the sliding component and the receiving chamber are arcuate shaped and the movement is angular.

In embodiments, a threaded component locks the sliding component in position within the receiving chamber.

In embodiments, a device comprises a first component comprising an anterior facing surface and a posterior facing surface; a first claw and a second claw rotatably attached to opposing peripheral ends of the posterior facing surface wherein each claw is independently rotatable around the respective joint, wherein the independently adjustable claws are configurable to increase the device's surface area coverage of an epiphyseal rim of a vertebral endplate. Other objects and advantages of this invention will become readily apparent from the ensuing description.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 shows illustrations of examples of dimensions available for a known medical Cage System (Kyocera S128 PEEK ALIF).

FIG. 2 shows experimental data published by Grant et al.

FIG. 3 shows endpoint morphology as documented by Wang, Y., et al., (2012) A morphological study of lumbar vertebral endplates: Radiographic, visual and digital measurements. European Spine Journal, 21(11), 2316-2323. Panel A shows an image of a vertebral endplate with identified epiphyseal rim and porous central core. Panel B shows graphs of shape and variations among vertebral segments.

FIG. 4 shows a chart of the variability in the thickness of the epiphyseal rim as adapted from Wang, Y., et al.

FIG. 5 shows illustrations of embodiments of the invention. For example, the top panel provides an embodiment where the arms are connected to the body with revolute joints. For example, the middle panel (top view) and bottom panel provides an embodiment wherein the implant comprises a single element with the arms rotating in light of the deformation of the joining portion.

FIG. 6 shows an illustration of an example of a spacer with integrated pockets and integrated lateral bodies.

FIG. 7 shows illustrations of examples of linking elements which can be anchored at the periphery of the lateral bodies (panel A) or in the center (panel B). In panel A, for example, the two bodies are connected by two deformable elements. Accordingly, a given distance between the body is obtained through plastic deformation of the connecting elements. In panel B, the two bodies are connected through two transverse elements that have two opposite threads, so that the rotation of a threaded rod placed in the two holes reduces or increases the distance with resultant lateral expansion or contraction of the implant.

FIG. 8 illustrates embodiments of the invention in a contracted state (panel A), in an expanded state (panel B), and with lateral bodies oriented at various angles (panel C). The single body can rotate in angles from 0° to 60°. The relative angle between the two bodies can be high as 150°.

FIG. 9 shows an illustration of embodiments of a sliding mechanism of the invention which allows for transversal expansion.

FIG. 10 shows an illustration of embodiments of the invention composed of multiple bodies joined to form a closed loop that can expand through gears. Panel A shows an isometric view, panel B shows a top view, panel C shows an example of gears to control expansion, and panel D shows a cross-section of the examples with gears.

FIG. 11 shows illustrations of embodiments of the invention. Panel A shows a body, such as a body made of titanium, that is hollow to host biologics. Panel B shows a body composed by structures imitating bone trabeculae. Panel C shows a body with host inserts in other material such as PEEK. Panel D shows an illustration of function as inner frame for bodies made of other material such as PEEK. Panel E shows a body made with a porous structure or a of a porous material.

FIG. 12 shows illustrations of embodiments of the invention wherein embodiments can also be provided with elements such as screws, blades, or plates to anchor to the surrounding bones.

FIG. 13 shows how the expansion of the invention can be actuated or maintained through treaded elements disposed internally between the bodies (panel B) or between the linking elements (panel A).

FIG. 14 shows manufactured components of embodiments of the invention. A shows the linking element that articulates with the arms show in B or the arms shown in C. In this configuration the variation in shape is obtained rigidly rotating the arms around the two respective hinges. Panels B and C are different geometrical shapes of the arms; panel D is an embodiment of the implant but without a wall delimiting the space for biologics; panels E and F are embodiments of the implant that are in contracted or expanded configuration; panels G and H are embodiments of the implant that are in expanded or contracted configurations (expansion as demonstrated in panel G is through the insertion of two threaded elements (not shown) that push against the arm to expand it outward); panels I and J are embodiments of the implant that can contract and/or expand through the insertion of a threaded element not shown; panels K and L are embodiments of the implant in which the threaded element acts against the two arm instead of against the two lateral bodies; panels M and N are embodiments of the implant that are in contracted or expanded; panels O and P are embodiments of the implant that provide holes to control contraction of the implant during manufacturing; panels Q and R provides an embodiment of the implant with a threaded element added to preserve the relative position of the connecting elements; panels S, T and U are embodiments of the implant as described herein; panel V and W are embodiments of the implant are the implant wherein the lateral expansion is “tutored” or “guided by a connecting element and is maintained through the interference screw in the middle; Panel X shows cage 13 compressed; Panel Y shows cage 14 compressed (6 mm gain); Panel Z shows cage 14 engaged; Panel AA shows cage 14 engaged with an interposed threaded element (considering that there is only one thread direction the hole close to the screw head is not threaded so the closing of the gap between the two connecting elements is obtained with the compression of the screw head against the connecting element); Panel AB shows cage 14 expanded; Panel AC shows a comparison of cage 6a.

FIG. 15 shows schematics of the configurations that include epiphyseal rim contact (panel A) and no epiphyseal rim contact (panel B).

FIG. 16 shows an image of mechanical testing using the Instron Mechanical testing system.

FIG. 17 shows a graph of non-limiting, exemplary results of measured stiffness of two constructs. Stiffness values [N/mm] of the constructs in relation to the position of the ALIF cage simulated through the intender.

FIG. 18 shows an illustration the expansion linear or along a curved path.

FIG. 19 shows a picture of a prototype at its minimal and expanded width.

FIG. 20 shows a CT reconstructed vertebra (panel a) and its cross section (panel b) at a given density threshold.

FIG. 21 shows the cross section of adjacent vertebral endplates (panel a) and the loft surface created by blending the two endplates (panel b).

FIG. 22 shows illustration of an implant created through the interpolation of the cross-sections of two adjacent vertebrae here shown with a tubular void to host biologics (panel a) and also a void created interpolating the profiles of high-density bone seen in the cross-sections (panel b).

FIG. 23 shows embodiments of the invention. Panel (A) shows an embodiment wherein the posterior wall is chosen at time of implantation in various length or cut to length and placed in sockets to preserve the volume allocated for biologics. Panels (B) and (C) shows embodiments wherein the posterior wall is shaped with the profile of squeezebox to limit the bending of the wall.

FIG. 24 shows embodiments of the invention. Panel (A) shows an embodiment wherein the posterior wall is sectioned to easy the flexion while preserving its resistance to compression. Panel (B) shows an embodiment wherein the posterior wall is made by two elements that slides on each other. Panel (C) shows an embodiment wherein the biologics can be hosted in a sack that can be hinged anteriorly. Expansion of the implant results in a smaller variation in the posterior shape of the implant.

FIG. 25 shows experimental testing, under an embodiment.

FIG. 26 shows experimental results in terms of subsidence load (a) and construct stiffness (b), under an embodiment.

FIG. 27 subsidence loads (a) and Construct Stiffness in relation to the cortical surface area of the bone surrogates(b), under an embodiment.

### DETAILED DESCRIPTION OF THE INVENTION

Presently, the intervertebral disk of a subject can be removed, and in its place, a “spacer” can be interposed between adjacent vertebrae to reestablish a vertebral spacing characteristic of the healthy spine segment. Failure of interbody spacers can be documented in the form of subsidence, especially in patients with osteoporosis, for which interbody fusion is usually contraindicated. However, subsidence of interbody spacers has been documented in percentage high as 76.7% (Choi & Sung, 2006).

Detailed descriptions of one or more embodiments are provided herein. It is to be understood, however, that the present invention can be embodied in various forms. Therefore, specific details disclosed herein are not to be interpreted as limiting, but rather as a basis for the claims and as a representative basis for teaching one skilled in the art to employ the present invention in any appropriate manner.

The singular forms “a,” “an,” and “the” include plural reference unless the context clearly dictates otherwise. The use of the word “a” or “an” when used in conjunction with the term “comprising” in the claims and/or the specification can mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises,” “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

As used herein, the term “about” can refer to approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

For purposes of the present disclosure, it is noted that spatially relative terms, such as “up,” “down,” “right,” “left,” “beneath,” “below,” “lower,” “above,” “upper” and the like, can be used herein for ease of description to describe one element or feature's relationship to another element(s) or feature(s) as illustrated in the figures. The spatially relative terms are intended to encompass different orientations of the device in use or operation in addition to the orientation depicted in the figures. For example, if the device in the figures is turned over or rotated, elements described as “below” or “beneath” other elements or features would then be oriented “above” the other elements or features. Thus, the exemplary term “below” can encompass both an orientation of above and below. The device can be otherwise oriented (rotated 90 degrees or at other orientations) and the spatially relative descriptors used herein interpreted accordingly.

As used interchangeably herein, “subject,” “individual,” or “patient,” can refer to a vertebrate, preferably a mammal, more preferably a human. In certain embodiments, “subject,” individual,” or “patient” refers to a reptile. Mammals include, but are not limited to, murines, simians, humans, farm animals, sport animals, and pets. The term “pet” includes a dog, cat, guinea pig, mouse, rat, rabbit, ferret, snake, turtle, lizard, bird, and the like. The term farm animal includes a horse, sheep, goat, chicken, pig, cow, donkey, llama, alpaca, turkey, and the like.

### Interbody Fusion Device

Aspects of the disclosure comprise, for example, an improved interbody fusion device. For example, embodiments of the interbody fusion device described herein can conform to the vertebral endplate and reduce the risk of implant subsidence, limit the number of templating attempts needed since the surgeon can fine tune the dimensions of the implant to the patient's anatomy after positioning, within the patient reduce the size of the incision needed since it can be expanded after positioning within the patient, and limit the number of implants need at the surgical site. As used herein, the terms “interbody fusion device” and “interbody cage” can be used interchangeably.

In embodiments, the interbody fusion device can comprise at least two lateral bodies configured to be in contact with a vertebral endplate of a subject.

The lateral bodies can comprise a smooth, semi-smooth, or rough surface. To increase friction, the bodies can be covered of texturing elements in shape of cones, spheres, or saw blade teeth to easy the insertion of the device while preventing its spontaneous removal. The bodies can further have a trapezoidal section to create concave of convex implant profiles. the Antero-posterior development of the bodies can be tapered at various angles to recreate various lordotic angles. The bodies can be implanted for interposition between the vertebrae or cemented

In embodiments, the lateral bodies can be any length (anterior-posterior) from about 20 mm to about 40 mm. In embodiments, the lateral bodies can be any width (medial-lateral) from about 6 mm to about 20 mm. In embodiments, the lateral bodies can be any thickness from about 5 mm to about 25 mm. But embodiments are not limited to these dimensions and length, width, and/or thickness may be greater or less.

In embodiments, the lateral bodies can be connected by at least one linking element. The term “linking element” can refer to a component and/or section of the fusion device which connects bodies of the fusion device, such as the lateral bodies of the fusion device. In embodiments, the linking element can be located at the periphery of the lateral bodies or at the center of the lateral bodies. For example, linking bodies located at the periphery of the lateral bodies increase volume available for biologics or cement, wherein linking bodies located at the central of the lateral bodies provides an option for surgeons working with patients for which the potential for intraforamina migration of the implant can be of concern.

In some embodiments, the linking elements can be curved or straight.

The components of the interbody fusion device, such as the lateral bodies and the linking element(s), can be connected in a variety of orientations, each of which can confer structural/functional properties to the device, including flexibility, functionality, durability, bioactive agent release characteristics, biocompatibility, availability, and cost.

In embodiments, the linking element can comprise a rigid element, a deformable element, or a combination thereof. The term “rigid” can refer to an element that is stiff or remains the same or similar to its original form after a force or pressure has been applied to it. The term “deformable” can refer to an element that can change in size or shape.

For example a linking element can have different thicknesses, so the deformation of the thick portions is marginal compared to thin portions. As such, the thick portions is then subject to a rigid movement that does not really represent a deformation. Similarly they can be made of materials that exhibit a visible deformation upon a certain load and become rigid after a certain stress. This is characteristic of material with fibers that show strong increment in stiffness when the fibers are all coherently aligned.

In embodiments, deformation can comprise elastic deformation and plastic deformation. The term “elastic deformation” can refer to a reversible change in shape or size of an object when a stress, force, and/or pressure is applied. The term “plastic deformation” can refer to a permanent mechanical deformation.

In embodiments, elastic deformation can be accomplished by the interbody fusion device further comprising a tensioning element. The term “tensioning element” can refer to an element which can be used to adjust, transmit, and/or secure tension. For example, the tensioning element can be interposed between two lateral bodies, linking elements, or a combination thereof. Non-limiting examples of tensioning elements can comprise a threaded element, a rack and pinion, a spring a self-locking bar, a self-locking bar with sawtooth profile, a press-fit post, or a combination thereof.

In embodiments, the linking element, the lateral bodies, or the lateral bodies connected by at least one linking element can be manipulated, such as to increase the surface area of the contact of interbody fusion device with the vertebral endplate. For example, the linking element, the lateral bodies, or the lateral bodies connected by at least one linking element can be manipulated at the time of plantation to match the epiphyseal rim of the vertebral endplate. The term “manipulate” can refer to physically altering the interbody fusion device described herein, such as altering the device's shape, position, size, or movement around a central axis. For example, device manipulation can be by deformation, displacement, or deflection. For example, the manipulation can comprise expansion, contraction, translation, and/or rotation. In embodiments, the manipulation can be in an x, y, and/or z coordinate system.

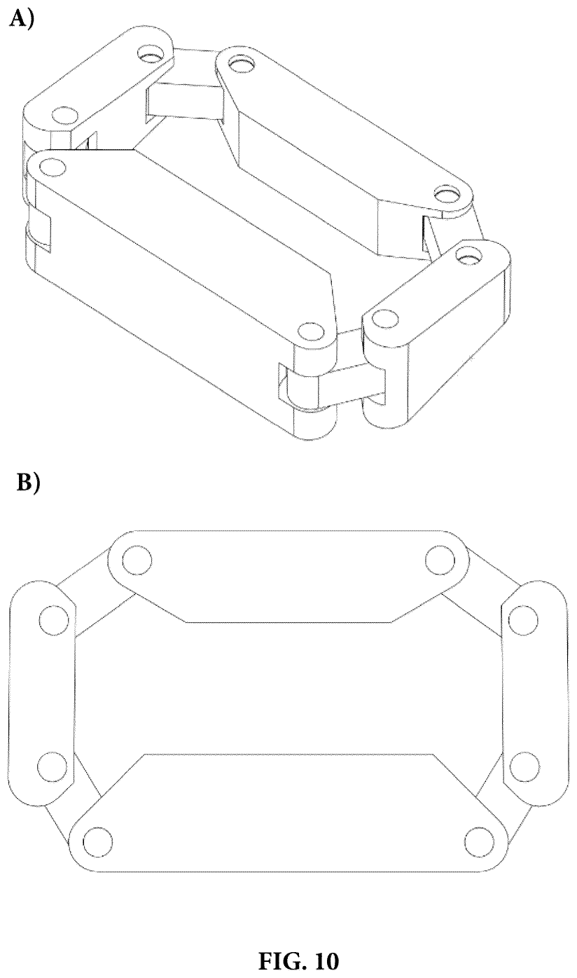

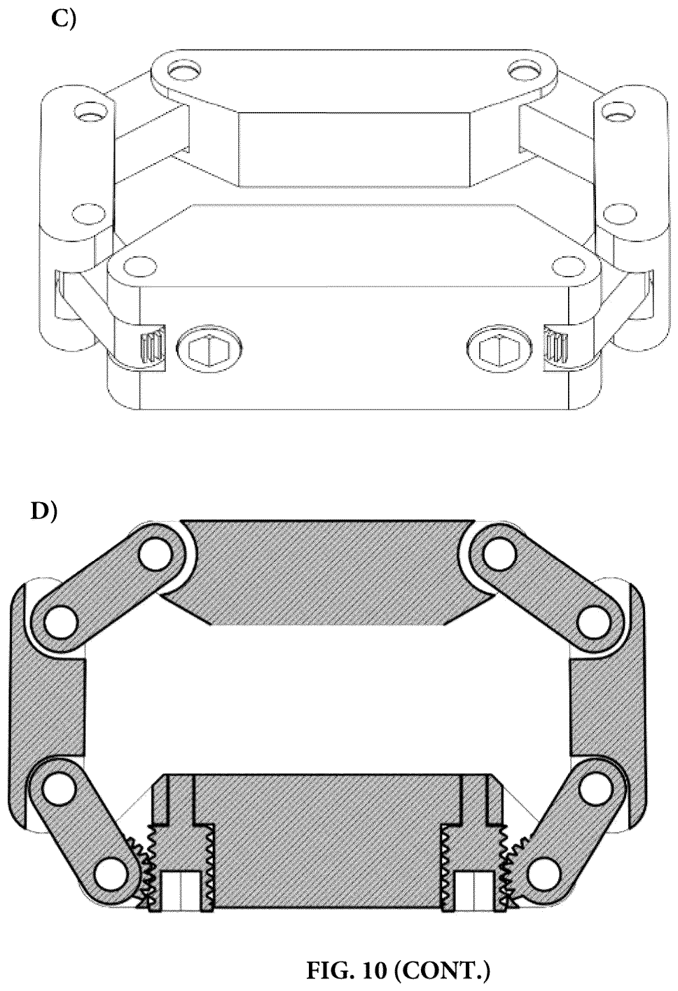

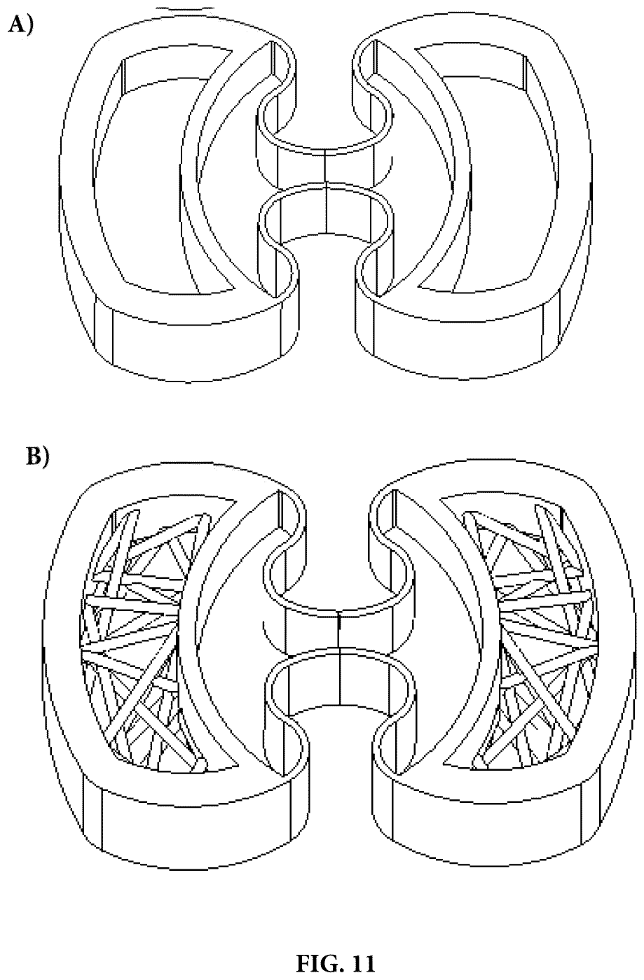

In embodiments, the interbody fusion device can comprise one or more elements that link lateral bodies and/or other elements of the interbody fusion device together. As used herein, the elements that link lateral bodies and/or other elements of the interbody fusion device together can be referred to as a “linking element”. For example, the linking element can be a sliding mechanism, a joint, a gear, or a combination thereof. For example, FIG. 9 shows an embodiment comprising a sliding mechanism and FIG. 10 shows an embodiment with multiple lateral bodies joined together by gears, thereby forming an expandable device.

In embodiments, the interbody fusion device can comprise an interior space enclosed or partly enclosed within the lateral bodies and/or linking elements. In embodiments, manipulation or deformation of the interbody fusion device can form an interior space. In embodiments, the linking interior space can be transversally delimited.

The interior space described herein can be configured to host a biologic or other active agent. Non-limiting examples of which include Hydroxyapatite, Magnesium, Chitosan, or a Hydrophilic coating. See, for example,

The term “biologic” can refer to a substance derived from a natural or biological source. For example, the biologic can comprise tricalcium phosphate, a bone morphogenetic protein, demineralized bone matrix, a stem cell, cortical or cancellous crushed bone, or a combination thereof.

In embodiments, a coating can be applied to the interbody fusion device or components thereof. The coating can be applied to a device in any suitable fashion, e.g., it can be applied directly to the surface of the medical device, or alternatively, to the surface of a surface-modified medical device, by dipping, spraying, or any conventional technique. The method of applying the coating composition to the device is typically governed by the geometry of the device and other process considerations. The coating can be a hydrophilic coating.

In embodiments, the device can be anchored to a subject, for example, anchored to the bone of a subject by a bone anchoring element. Non-limiting examples of bone anchoring elements can comprise a screw, a blade, a plate, or a combination thereof.

In other embodiments, the device can be left in place without anchoring.

In embodiments, the interbody fusion device or components thereof, such as the lateral bodies and/or the linking elements, can comprise a biocompatible material, e.g., such that it results in no induction of inflammation or irritation when implanted. The term “biocompatible” can refer to a material which is not toxic, not injurious or not inhibitory to mammalian cells, tissues, or organs with which it comes in contact. Furthermore, when the material is in use with respect to the interbody fusion device, it does not induce an immunological or inflammatory response sufficient to be deleterious to the subject's health or to implantation of the scaffold. Non-limiting examples of materials that embodiments can comprise include a photoresist polymer or a polymer that is compatible with three-dimensional printing technology, such as (2-(Hydroxymethyl)-2-[[(1-oxoallyl)oxy]methyl]-1,3-propanediyldiacrylate, known as IP-L; Photoresist pentaerythritol tetraacrylate (PETTA, Sigma-Aldrich) containing 3% Irgacure 379 photoinitiator (Ciba); polyurethanes polycaprolactone, polyglycolic acid, polylactic acid, polyamides, polyolefin, polyester, polytetrafluoroethylene, polyurethanes, and hydrogels used for bioprinting such as collagen, alginate, agarose, and chitosan, and synthetic hydrogels such as hyaluronan-methylcellulose, polyethylene glycol diacrylate collagen, laminin, matrigel, and non-biodegradable materials such as polysiloxanes, Stainless steel, Co—Cr alloys, Ti-alloys.

In embodiments, the interbody fusion device or components thereof comprise fiber, nylon, titanium, polyether ether ketone (PEEK), a hydroxyapatite (HA)-PEEK composite, a titanium-PEEK composite, polyethylene, polyethylene composite, stainless-steel, a stainless-steel composite, cobalt-chrome, a cobalt-chrome composite, tantalum, a tantalum composite, ceramic, a ceramic composite, an alloy, a composite, or a combination thereof.

In embodiments, the interbody fusion device or components thereof, such as the lateral bodies and/or the linking elements, can comprise a solid material, a semi-solid material, or a porous material.

The term “porous” can refer to a substance that contains pores and/or is permeable.

The term “non-porous” can refer to a material which is not penetrable by liquids or gases and/or does not contain pores.

In embodiments, the solid material can comprise comprises titanium, polyether ether ketone (PEEK), a hydroxyapatite (HA)-PEEK composite, a titanium-PEEK composite, polyethylene, polyethylene composite, stainless-steel, a stainless-steel composite, cobalt-chrome, a cobalt-chrome composite, tantalum, a tantalum composite, ceramic, a ceramic composite, or a combination thereof. For example, the material can be medical grade stainless steel, titanium alloy, polylactic/polyglycolic acid, polyether ether ketone (PEEK), methyl methacrylate, and the like.

The term “composite” can refer to a material comprising two or more materials having different physical or chemical properties from each other, wherein the composite has different properties from the individual materials comprising the composite, and wherein the individual materials are macroscopically or microscopically separated and distinguishable from each other in the final structure of the composite. In embodiments, a composite can refer to different materials that are mechanically joined together or manufactured intertwined.

The term “alloy” can refer to a substance composed of two or more metals or of a metal and a nonmetal intimately united, for example by chemical or physical interaction. Alloys can be formed by various methods, including being fused together and dissolving in each other when molten, although molten processing is not a requirement for a material to be within the scope of the term “alloy.” Alloys can have physical or chemical properties that are different from its components. In embodiments, the terms “composite” and “alloy” can be used interchangeably.

In embodiments, materials as described herein can imitate biological tissues and/or bone, such as bone trabecula. As used herein, the term “imitate” can refer to mimicking or assuming the appearance and/or properties thereof.

In embodiments, the interbody fusion device can be adapted for in vivo use, such as for implantation into a subject. In presence of large tissue defects, such as large defects of the bone, tendons, ligaments, cartilage, and the interbody fusion device can serve as a matrix for the regeneration of tissue. For example, the interbody fusion device can store and/or release bioactive agents, nutrients, water, cell survivability enhancers and/or growth factors; provide mechanical stimuli; and/or induce cell proliferation.

In embodiments, the interbody fusion device is constructed so as to avoid immunological responses (i.e., biocompatible) such that it results in no induction of inflammation or irritation when implanted into a subject.

In other embodiments, the interbody fusion device can be constructed of a biodegradable materials so as to degrade over a period of time, for example as cells proliferate and/or tissue regenerates. In other embodiments, the interbody fusion device can be constructed of non-biodegradable materials.

As described herein, the interbody fusion device can host cells and nutrients so as to assist and/or promote tissue regeneration. For example, the interbody fusion device can serve as a foundation upon which cells can adhere to and proliferate, thus assisting in the regeneration of tissue.

In embodiments, the interbody fusion device can be seeded with viable cells so as to populate the bioscaffold with the viable cells. The term “viable cell” can refer to a cell that is alive and capable of growth, proliferation, migration, and/or differentiation. The interbody fusion device can act as structural scaffold upon which viable cells can migrate and readily repopulate. In some embodiments, cells from the native tissue (e.g., the host subject) can also migrate into the interbody fusion device and readily repopulate the polymer-permeated graft in vivo.

For example, the interbody fusion device can be seeded and incubated with exogenous cells under conditions conducive to populating the bioscaffold with the exogenous cells or cells derived from the exogenous cells. In some embodiments, the exogenous cells can be autologous, homologous (e.g., allogenic), or heterologous. For example, “autologous” refers to biological material (e.g., exogenous cells) that will be introduced into the same individual from whom the material was collected or derived. For example, “homologous” can refer to biological material (e.g., exogenous cells) collected or derived from a compatible donor that will be introduced into a different individual from which the material was collected or derived. For example, “heterologous” can refer to biological material (e.g., exogenous cells) collected or derived from a compatible donor of a different species that will be introduced into an individual. Non-limiting examples of cells that can be seeded onto (and thus useful for populating the interbody fusion device) include osteoblasts, osteoclasts, lining cells, keratinocytes, melanocytes, nerve cells, stromal cells, fibroblasts, endothelial cells, progenitor cells, stem cells, organ-specific cells, tissue-specific cells, or a combination thereof.

Conditions conducive to populate the interbody fusion device are dependent upon the cells used, and can include temperature, the presence or absence of growth factors, the presence or absence of differentiation factors or migration factors, or the air content. In embodiments, the interbody fusion device is introduced or implanted into a subject, and the subject's own cells migrate into the device. In other embodiments, viable cells are introduced into the interbody fusion device prior to implanting the graft onto the subject.

One of skill in the art can seed exogenous cells onto the interbody fusion device by placing the interbody fusion device into culture medium containing dissociated, or dissociated and expanded, cells and allowing the cells to migrate into the interbody fusion device and populate the interbody fusion device. In some embodiments, cells can be injected into one or more places in the interbody fusion device, such as into the interior, in order to accelerate repopulation of the structures.

The viable cells can be cultured prior to populating or seeding of the interbody fusion device. Culture mediums used to grow and expand cells of interest is cell-type-dependent, and is known to those skilled in the art. The culture medium can be serum-free and would not require the use of feeder cells.

The interbody fusion device can comprise at least one bioactive agent, which can refer to virtually any substance which possesses desirable characteristics for application to the implant site. For example, the interbody fusion device can be coated with the at least one bioactive agent or can contain the bioactive agent so as to release the bioactive agent within a subject. The bioactive agents useful in the present invention include thrombin inhibitors, antithrombogenic agents, thrombolytic agents, fibrinolytic agents, vasospasm inhibitors, calcium channel blockers, vasodilators, antihypertensive agents, antimicrobial agents, antibiotics, inhibitors of surface glycoprotein receptors, antiplatelet agents, antimitotics, microtubule inhibitors, anti-secretory agents, actin inhibitors, remodeling inhibitors, antisense nucleotides, anti-metabolites, antiproliferatives, anticancer chemotherapeutic agents, anti-inflammatory steroid or non-steroidal anti-inflammatory agents, immunosuppressive agents, growth hormone antagonists, growth factors, vitamins, cell viability enhancers, dopamine agonists, radiotherapeutic agents, peptides, proteins, enzymes, extracellular matrix components, ACE inhibitors, free radical scavengers, chelators, antioxidants, anti-polymerases, antiviral agents, photodynamic therapy agents, and gene therapy agents.

“Cell Viability Enhancer” can refer to a substance that enhances and/or promotes the viability and/or growth of a cell.

“Antibiotic” can refer to a substance that controls the growth of bacteria, fungi, or similar microorganisms, wherein the substance can be a natural substance produced by bacteria or fungi, or a chemically/biochemically synthesized substance (which may be an analog of a natural substance), or a chemically modified form of a natural substance. One of skill will recognize that the scaffold can be coated with a wide variety of antibiotics, such as penicillins, cephalosporins, macrolides, fluoroquinolones, sulfonamides, tetracyclines, aminoglycosides, and the like.

The interbody fusion device can have the structural, mechanical and functional properties described herein while at the same time be able to withstand physiological loads. For example, embodiments can comprise new three-dimensional scaffolds for extended tissue reconstruction that are able to withstand physiological loads. In embodiments, the interbody fusion device demonstrates enhanced cell survivability.

The interbody fusion device can be manufactured using techniques known in the art, such as utilizing three-dimensional printing or assembling pre-built components, such as pre-built lateral bodies and linking elements.

Embodiments described herein can be produced using 3D printing, injection molding manufacturing, or a combination thereof. For example, the 3D printer can print the device as titanium, polyether ether ketone (PEEK), a hydroxyapatite (HA)-PEEK composite, a titanium-PEEK composite, polyethylene, polyethylene composite, stainless-steel, a stainless-steel composite, cobalt-chrome, a cobalt-chrome composite, tantalum, a tantalum composite, ceramic, a ceramic composite, or a combination thereof. For example, the material can be medical grade stainless steel, titanium alloy, polylactic/polyglycolic acid, polyether ether ketone (PEEK), methyl methacrylate, and the like. The material can be composed of about 10%, 20%, 30%, 40%, 50%, 60%, 70%, 80%, 90%, or 100% of a biocompatible material.

In embodiments, the 3D printed device can be printed to conform with an individual patient's anatomy to reestablish a vertebral spacing characteristic of the healthy spine segment.

Aspects of the invention are also directed towards a method for designing the interbody fusion device. In embodiments, the methods can be provided as a computer-aided-design. See, for example, U.S. Pat. No. 7,747,305, which is incorporated by reference herein in its entirety. Such computer aided design methods can relate to the generation of patient-specific and/or tissue-specific interbody fusion devices for in vivo use. In embodiments, the method can be provided as an algorithm. The method can receive input manually (such as 3D geometries designed in a CAD or coordinates given in input to define geometric primitives) and/or from one or more elements obtained from diagnostic imaging, such as reconstructions of bone, blood vessels, material density, and structural properties distributions. Embodiments can further comprise the input of elements as copy of existing reconstruction that can be used on a specific patient with scaling or/and morphing, for example, as is the case of scaffold applied to subject A but obtained from CT data of subject B.

### Methods of Use

Aspects of the invention are drawn to methods for treating, preventing, or delaying spinal pathologies by implanting in the subject embodiments of the interbody fusion device as described herein.

The term “subject” or “patient” can refer to any organism to which aspects of the invention can be administered, e.g., for experimental, diagnostic, prophylactic, and/or therapeutic purposes. Typical subjects to which compositions of the present disclosure can be administered will be mammals, particularly primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals particularly pets such as dogs and cats. For diagnostic or research applications, a wide variety of mammals will be suitable subjects, including rodents (e.g., mice, rats, hamsters), rabbits, primates, and swine such as inbred pigs and the like. The term “living subject” can refer to a subject noted above or another organism that is alive. The term “living subject” can refer to the entire subject or organism and not just a part excised (e.g., a liver or other organ) from the living subject.

The term “treating” can refer to partially or completely alleviating, ameliorating, improving, relieving, delaying onset of, inhibiting progression of, reducing severity of, and/or reducing incidence of one or more symptoms, features, or clinical manifestations of a particular disease, disorder, and/or condition. Treatment can be administered to a subject who does not exhibit signs of a disease, disorder, and/or condition (e.g., prior to an identifiable disease, disorder, and/or condition), and/or to a subject who exhibits only early signs of a disease, disorder, and/or condition for the purpose of decreasing the risk of developing pathology associated with the disease, disorder, and/or condition.

The term “preventing” can refer to protecting against the development or a disease or disorder. For example, prevention to refer to precluding, averting, obviating, forestalling, stopping, or hindering a disease, disorder, or symptom thereof.

The term “delaying” can refer to the act of postponing, hindering, or causing something to occur more slowly than normal. For example, embodiments as described herein can delay the onset of symptoms of a spinal pathology.

The term “spinal pathology” can refer to the anatomic and physiological deviations from a normal spine that constitute disease or characterize a particular disease.

In embodiments, the spinal pathology can be a spinal disease. The term “disease” can refer to a disorder of structure or function. Non-limiting examples of spinal diseases that can be treated, prevented, or delayed by embodiments as described herein comprise degenerative disk disease, osteoporosis, spondylolisthesis, a herniated disk, spine stenosis, spondylosis, kyphosis, myelopathy, scoliosis, spinal deformities, spinal tumors, or a combination thereof. In embodiments, the interbody fusion device can be used on patients afflicted with osteoporosis.

In embodiments, the spinal pathology can be an injury. The term “injury” can refer to damage, harm, or loss as the result of external force. Non-limiting examples of spinal injuries that can be treated, prevented, or delayed by embodiments as described herein comprise trauma, degenerative diseases, tumors, stenosis, instability, anterolisthesis, retrolisthesis, lateral listhesis, scoliosis, flatback syndrome, post-diskectomy collapse, or post-laminectomy syndrome.

For example, the trauma can comprise blunt force trauma or penetrating trauma. In embodiments, the trauma can comprise a surgical procedure, a car accident, or a sports injury.

In embodiments, the spinal pathology can be a spinal deformity. The term “spinal deformity” can refer to a spine or portion thereof that does not have a natural and/or normal shape or form. Non-limiting examples of spinal deformities that can be treated, prevented, or delayed by embodiments as described herein comprise scoliosis, kyphosis, lordosis, anterolisthesis, retrolisthesis, lateral listhesis, hemivertebrae, post-traumatic deformity, or a combination thereof.

In embodiments, aspects of the invention provide enhanced stability of an interbody spacer. For example, the invention decreases the risk of subsidence and translation of an interbody spacer in a patient. The term “subsidence” can refer to sinking or settling in bone. For example, the subsidence can occur in a spacer during interbody fusion. For example, subsidence can occur when a spacer penetrates the vertebral endplate. Subsidence can typically occur over time or after surgery. For example, the interbody fusion device described herein can reduce the risk of subsidence of interbody spacers. Translation of the device is meant to indicate gross movement anterior, posterior, and/or lateral to the index placement or position.

Embodiments as described herein can be implanted in a subject before, during, or after a surgical procedure. For example, the procedure for installing embodiments of the fusion device as described herein is similar to known procedures for anterior, oblique, or lateral lumbar interbody fusion implantation. Accordingly, embodiments can only be implanted during surgery; however, embodiments can be utilized as part of a staged operation with multiple parts on the same patient.

The term “implant” or “implanted” can refer to the positioning of a medical device, such as the interbody fusion device to be positioned at a location within a body, such a spine. The terms “implantation” and “implanted” can refer to the positioning of a medical device at a location within a body of a subject.

Embodiments can comprise removing an interverbal disk, or portion thereof, from a subject afflicted with a disease, injury, or combination thereof, thereby providing an implant site; decorticating the endplate and/or removal of the cartilaginous endplate aligning the interbody fusion device with the vertebral endplate of a subject; and positioning the interbody fusion device within the implant site.

In embodiments, the implant can be placed parallel to the lordotic or kyphotic angle of one of the endplates or average angle between the two endplates. The implant can be placed as anterior or posterior at the discretion of the surgeon, but not beyond the posterior vertebral edge nor in the spinal canal. The implant is then expanded to maximize footprint, increase volume for biologics and/or epiphyseal rim contact. Once expanded, the implant is then locked into position. The expanded implant can be secured further with additional bony fixation such as screws, anchors, blades, pegs, and/or plate.

In embodiments, the method can comprise an addition step of manipulating the device so as to increase the surface area coverage of an epiphyseal rim of the vertebral endplate without exceeding the endplate footprint. Biologics can be placed within the invention prior to the implantation. Biologics and/or bone grafts can also be added to the invention after implantation via an opening through which biologics can be injected or impacted within. The addition of biologics will be performed as a combination of the above. Examples of delivery methods can be existing products such as: GraftGun® from SurGenTec, InstaFILL™ from Globus Medical, LITe® BIO from Stryker, and spineology bone slurry tubes.

In embodiments, the device can be left in place without further securing. In embodiments, the device can be secured. For example, the device can be anchored to the bone of the subject as described herein.

### Kits

Further, aspects of the invention provide for kits comprising the interbody fusion device described herein and instructions for use. For example, the kit can comprise one or more of the following components. For instance, the kit can comprise components useful for carrying out methods of the invention and instructional material that describes the method of utilizing the device as described herein.

In certain embodiments, the kits can comprise spacers of various sizes, shapes, and flexibilities, wherein the appropriate size is selected according to the needs of the patient, the preference of the doctor, or a combination thereof. Kit embodiments can further include a drill.

In certain embodiments, the kit comprises instructional material.

Instructional material can include a publication, a recording, a diagram, or any other medium of expression which can be used to communicate the usefulness of the device or kit described herein. The instructional material of the kit of the invention can, for example, be affixed to a package which contains one or more instruments which can be necessary for the desired procedure. Alternatively, the instructional material can be shipped separately from the package, or can be accessible electronically via a communications network, such as the Internet. The disclosures of each and every patent, patent application, and publication cited herein are hereby incorporated herein by reference in their entirety. While this invention has been disclosed with reference to specific embodiments, it is apparent that other embodiments and variations of this invention can be devised by others skilled in the art without departing from the true spirit and scope of the invention. The appended claims are intended to be construed to include all such embodiments and equivalent variations.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

### Comfortable Interbody Fusion Device

The invention herein disclosed comprises an interbody fusion device that can conform to the vertebral endplate in order to maximize the surface on contact and therefore reduce the risk of implant subsidence.

Non-Limiting Competitive Advantages: Without wishing to be bound by theory, embodiments of the invention can conform to the vertebral endplates. Such characteristic allows for maximization of the surface of contact between cage and high-density regions of the endplate with resultant reduction of subsidence risk (reduction of post-operative failures). Furthermore, since the device can be inserted in a contracted shape, if compared to standard anterior interbody devices, it can result also in a reduced dissection to access the site (reduction of inter-operative complications). In light of its characteristic to vary the dimensions, the invention can be implanted with reduced number of templating attempts (Reduction of surgical time) and can be distributed in a reduced number of sizes (Reduction of distribution costs).

### Background

For the treatment of multiple spinal pathologies, such as degenerative disk disease and spondylolisthesis, surgical treatment, such as the vertebral interbody fusion can be used. See, for example, U.S. Pat. Nos. 10,201,431 and 10,105,238. In this procedure, the intervertebral disk is removed, and, in its place, a “spacer” is interposed between adjacent vertebrae to reestablish a vertebral spacing characteristic of the healthy spine segment. The spacers can be designed in PEEK, Titanium, or a combination of the two and in some cases are able to be configurated with screws or blades to improve fixation to bone. In most of the cases, the spacers are porous with pockets to host biologics. Spacers can be constituted by a unique monoblock but in some cases, to reduce the dimension of the incision, these spacers are constituted by a plurality of articulating elements that are inserted in a compact envelope and expanded on site.

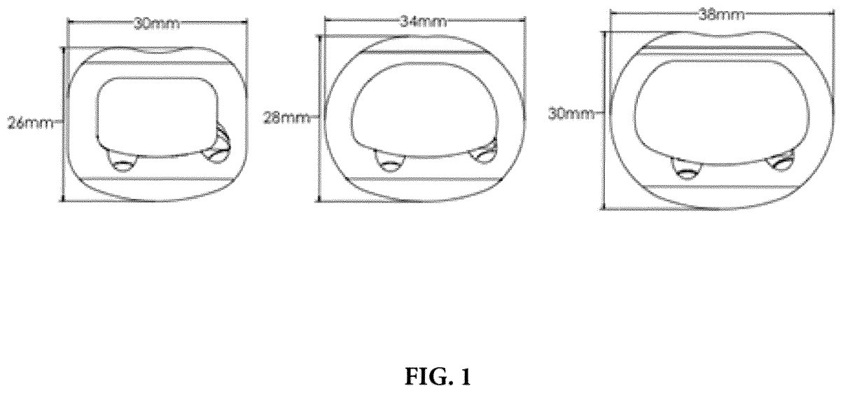

Failure of interbody spacers can be documented in the form of subsidence, for example in patients with osteoporosis, for which interbody fusion is can be contraindicated. Subsidence of interbody spacers has been documented in percentage high as 76.7% (Choi & Sung, 2006). To understand such phenomenon, Yuan et al. (Yuan, Kaliya-Perumal, Chou, & Oh, 2020) found that using bone surrogates with increased cage size correlates with the force required to cause subsidence. Surgeons attempt to maximize the cage size using existing intervertebral spacer that are distributed at discrete values of thickness, depth, and width in increments of few millimeters (see FIG. 1 as example).

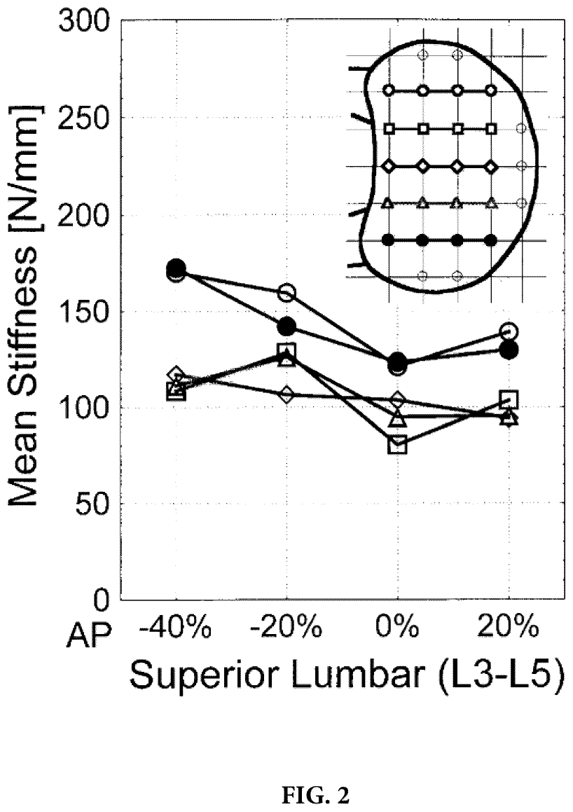

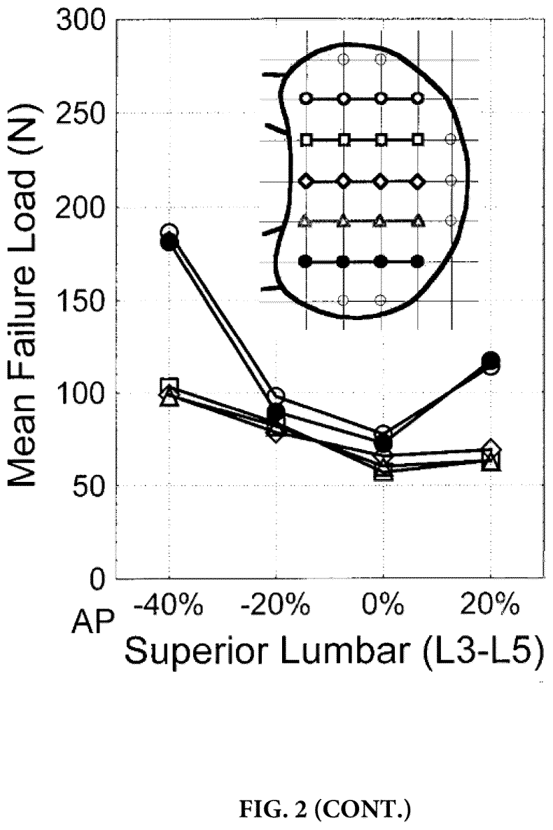

Stiffness and strength of the endplates have strong variability along the endplate as shown in the data provided by Grant et al. for Lumbar vertebrae, see FIG. 2 (Grant, Oxland, & Dvorak, 2001). More specifically stiffness is higher in the lateral than in the center portions while strength in the anterior and posterior lateral portions of the endplate is higher than in other regions.

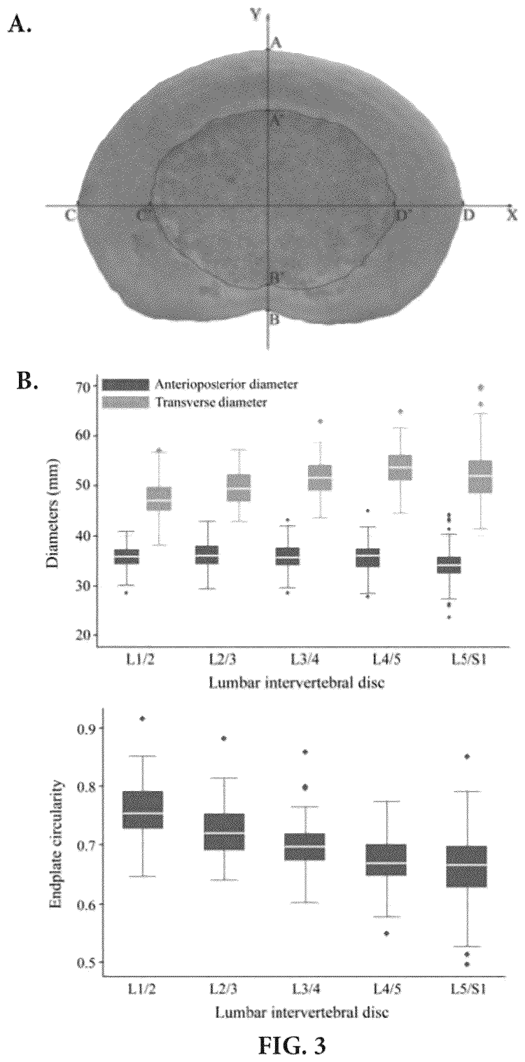

These portions of the endplate consist in the “epiphyseal rim” that is distinguished by a lower porosity than the central portion of the endplate, see FIG. 3 panel a taken from (Wang, Battié, & Videman, 2012). From the same study it has also been documented a strong variability in shape and dimensions among segments and subjects (see FIG. 3 panel b).

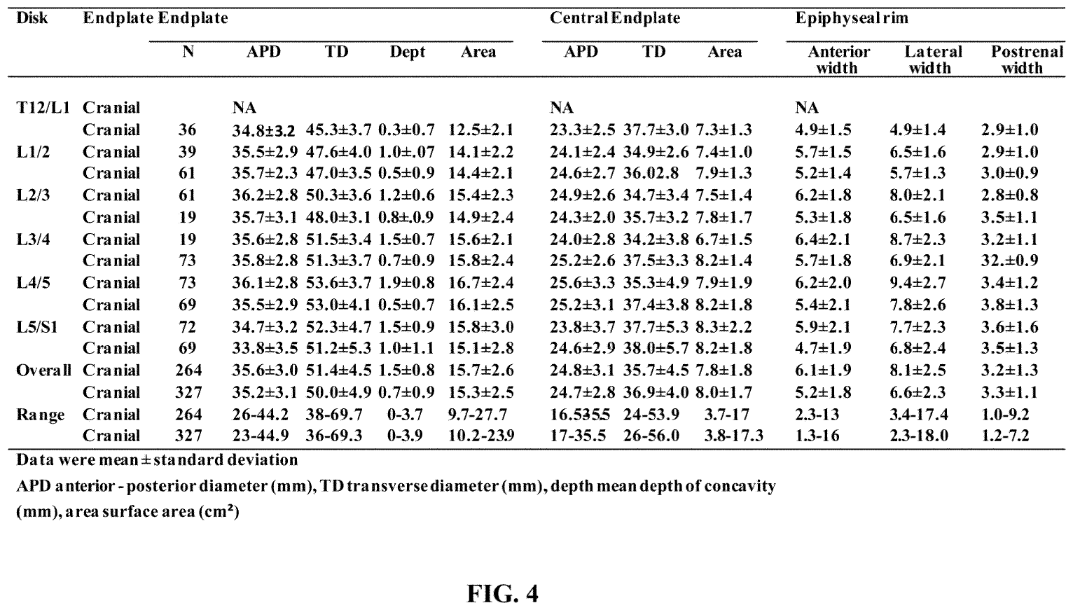

Strong variability in the thickness has been found of the epiphyseal rim as shown in FIG. 4, taken from Wang et al. Therefore, a cage conceived in line with existing devices that can fit the variability of the endplate shape and the thickness of the epiphyseal rim would require hundreds of configurations to be kept on the surgical site.

Moreover, a study of Phan and Mobbs (2016) highlighted that optimal cage dimensions are dictated by the anatomy, with cages that too small do not provide adequate stability while too large can damage surrounding structures (Phan & Mobbs, 2016).

The invention herein presented is a cage that can be tailored at time of insertion on the endplate dimensions to maximize the coverage of the epiphyseal rim.

### REFERENCES IN THIS EXAMPLE

Choi, J. Y., & Sung, K. H. (2006). Subsidence after anterior lumbar interbody fusion using paired stand-alone rectangular cages. European Spine Journal, 15(1), 16-22. doi.org/10.1007/s00586-004-0817-y
Grant, J. P., Oxland, T. R., & Dvorak, M. F. (2001). Mapping the structural properties of the lumbosacral vertebral endplates. Spine, 26(8), 889-896. doi.org/10.1097/00007632-200104150-00012
Phan, K., & Mobbs, R. J. (2016). Evolution of Design of Interbody Cages for Anterior Lumbar Interbody Fusion. Orthopaedic Surgery, 8(3), 270-277. https://doi.org/10.1111/os.12259
Wang, Y., Battid, M. C., & Videman, T. (2012). A morphological study of lumbar vertebral endplates: Radiographic, visual and digital measurements. European Spine Journal, 21(11), 2316-2323. doi.org/10.1007/s00586-012-2415-8
Yuan, W., Kaliya-Perumal, A. K., Chou, S. M., & Oh, J. Y. L. (2020). Does Lumbar Interbody Cage Size Influence Subsidence? A Biomechanical Study. Spine, 45(2), 88-95. doi.org/10.1097/BRS.0000000000003194

The present invention can relate to spine interbody fusion devices.

### Non-Limiting Summary of the Invention

Without wishing to be bound by theory, embodiments of the invention can have a mutable shape that can be finely adjusted in its dimensions to the endplate within a continuous range of dimensions.

Such characteristics allows:
1) Maximization of the epiphyseal rim coverage and therefore reduction of subsidence risk.
2) Limit the number of templating attempts needed since the surgeon can fine tune the dimensions of the implant after positioning.
3) Reduce the size of the incision needed since it can be expanded after positioning.
4) Limit the number of implants needed at the surgical site.

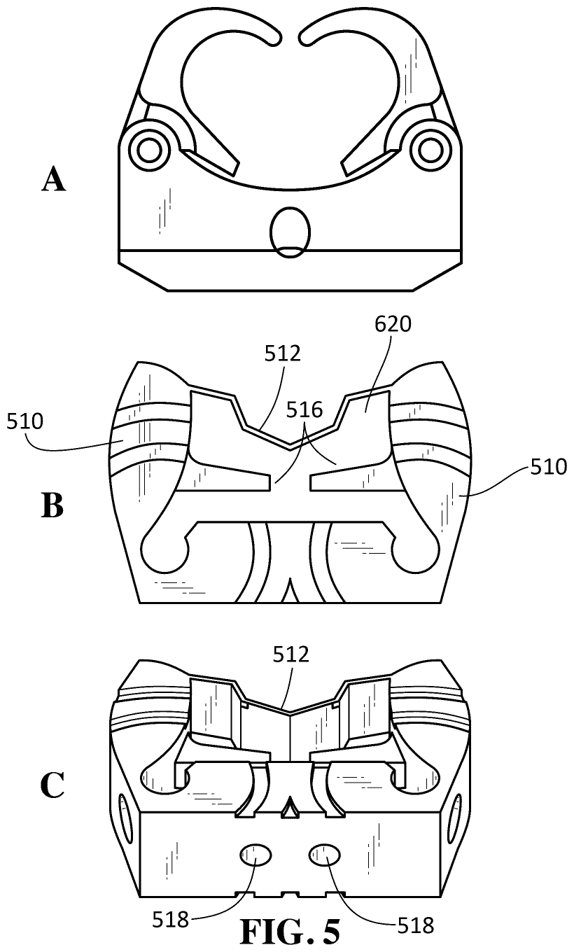

Embodiments described herein can be composed of two or more bodies (see FIG. 5) that are designed to be in contact with the surrounding endplates. These bodies are connected through linking elements that are configured at time of implantation to match the endplate. The linking elements can be rigid and connected through joints to the bodies (see FIG. 5, panel A) or can constitute a unique structure with the bodies and configured through deformation (FIG. 5, panels B and C). Such deformation can be within the elastic range for which a third tensioning element is interposed or can be performed within its plasticity range. These deformable elements can also be shaped to form a transversally delimited chamber able to host biologics to enhance bone formation within the intervertebral disk space. Under an embodiment, lever arms can be present to easy the expansion of the spacer actuated by threaded elements.

FIG. 5 (panel B) shows a top view of an interbody fusion device, under an embodiment. The device features lateral bodies 510 and linking element 512. Note that the linking element 512 and lateral bodies 512 are at least partially deformable. FIG. 5 (panel C) shows a perspective view of the same device and illustrates threaded through holes 518 on a surface (opposite the linking element 512). In operation, a user drives a threaded element through the openings until distal ends of the screws contact arms 516. As the threaded element advances position, a force is applied to the arms. As the arms and lateral bodies are integrally formed, the upward force causes an expansion of the linking element 512.

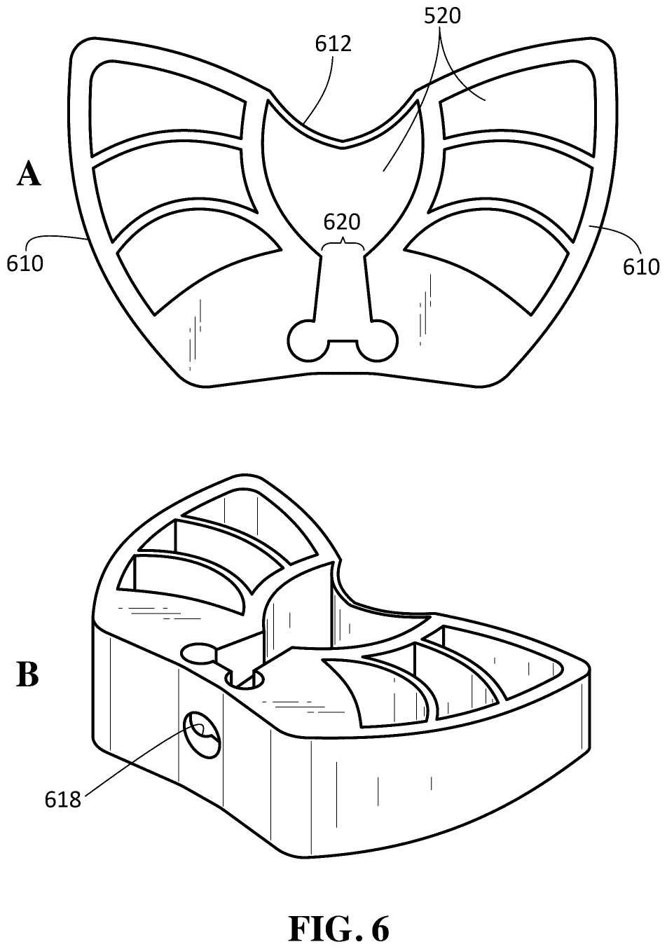

FIG. 6 (panel A) shows a top view of an interbody fusion device, under an embodiment. The device features lateral bodies 610 and linking element 612. Note that the linking element 612 and lateral bodies 610 are at least partially deformable. FIG. 6 (panel B) shows a perspective view of the same device and illustrates a threaded through hole 618 on its lower surface (opposite the linking element). In operation a screw is driven through the threaded opening thereby entering a passageway which narrows towards a gap 620. The screw is slightly larger in diameter than the opening. Accordingly, upward movement of the screw forces the opening to widen and thus causes expansion of the linking element. Note that the devices seen in FIG. 5 (panel A and panel B) and FIG. 6 (panel A and panel B) feature interior spaces 520, 560 for hosting biologics.

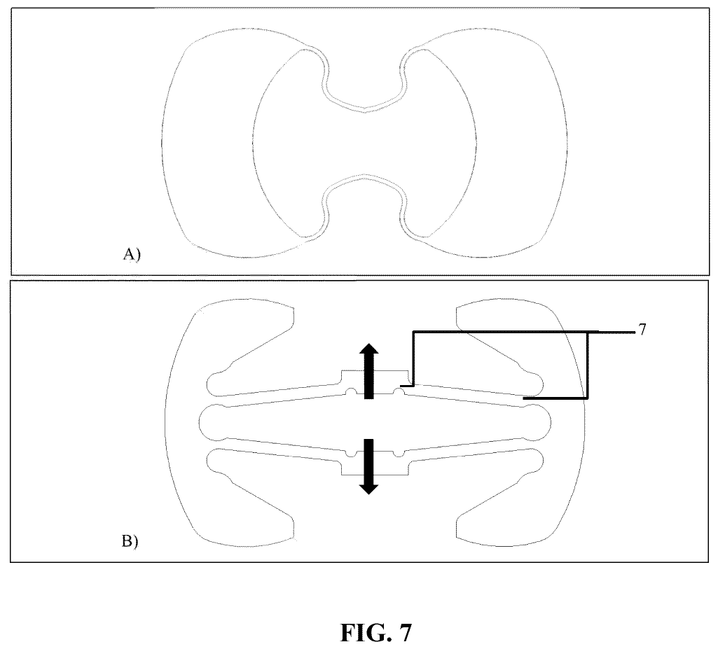

The linking elements can be anchored at the periphery of the lateral bodies (FIG. 7, panel A) or in the center (FIG. 7 panel B). In an embodiment of the invention (panel A), the linking elements are curved to accommodate large displacement between the bodies with limited local deformation of the elements.

In an additional embodiment (panel B) the linking elements deformation mainly happens in specific locations (7) to allow the rigid rotation of the elements to compose a quadrilateral expandable mechanism that can be expanded through the use of a threaded interposed element.

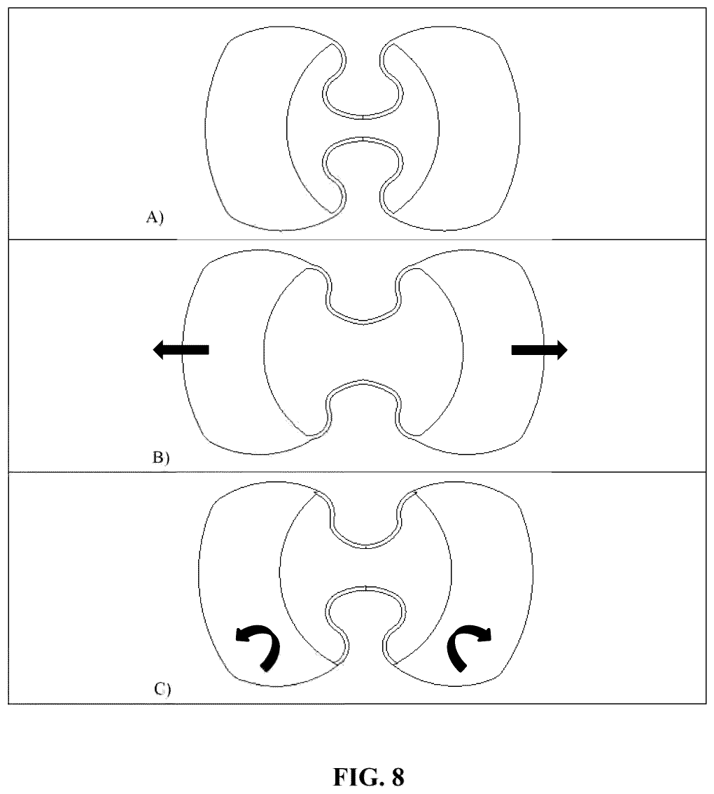

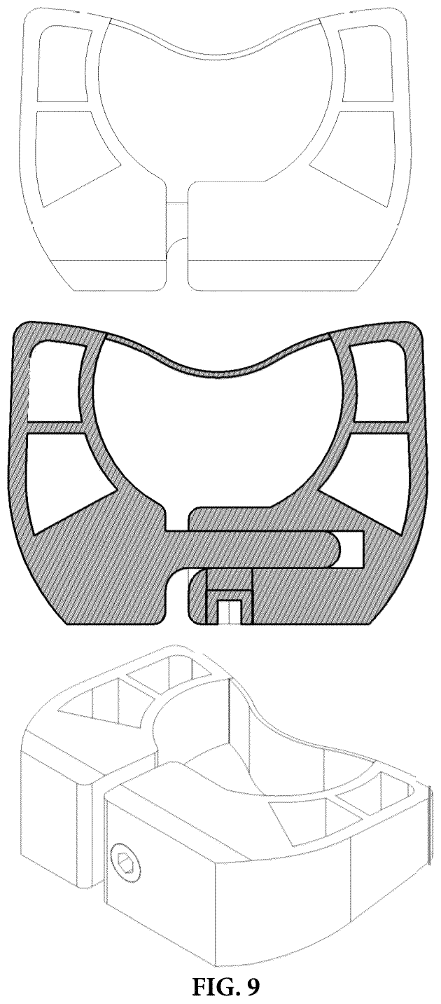

The invention, from its contracted state, see FIG. 8 (panel A) is configured to expand (panel B) and is also configurable with lateral bodies oriented at a certain angle. FIG. 8 illustrates embodiments of the invention in a contracted state (panel A), in an expanded state (panel B), and with lateral bodies oriented at various angles (panel C). A single body can rotate in angles from 0° to 60° where the angle represents an offset from a vertical axis of the single body in an undeployed state. Embodiments may allow a rotational angle greater than 60°.

The two bodies composing the invention can also be joined trough a sliding mechanism as shown in FIG. 9. Under an embodiment, the anterior sliding mechanism allows widening of the implant while maintaining rigidity to torsion while the posterior linking element delimits the chamber to host biologics and it can also provide transverse force to easy expansion or compression of the implant. Under this embodiment, with proper sizing of the posterior linking element, it is possible to keep it in an elastic range of deformation for which it works like a spring, providing force for expansion of contraction. However, embodiments are not so limited. Various configurations of the linking element with respect to this same design are described below.

An anterior screw can be added to secure the width of the implant.

An additional example of the invention is when the expansion is obtained through bodies connected with a series of joints as shown in FIG. 10. In this configuration, gears can be included in the design to actuate the expansion.

In additional embodiments, the bodies are made of Titanium and made hollow to host biologics, FIG. 10, panel A, are composed of structures imitating bone trabeculae (panel B), host inserts in other material such as PEEK (panel C), function as inner frame for bodies extensively made of other material such as PEEK (panel D), and are made with a porous structure or of a porous material (panel E).

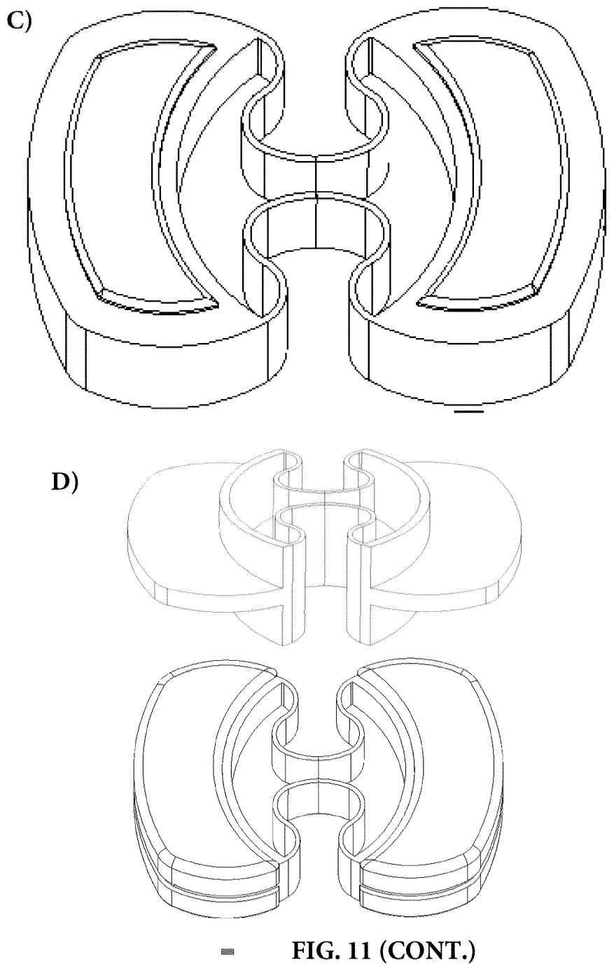

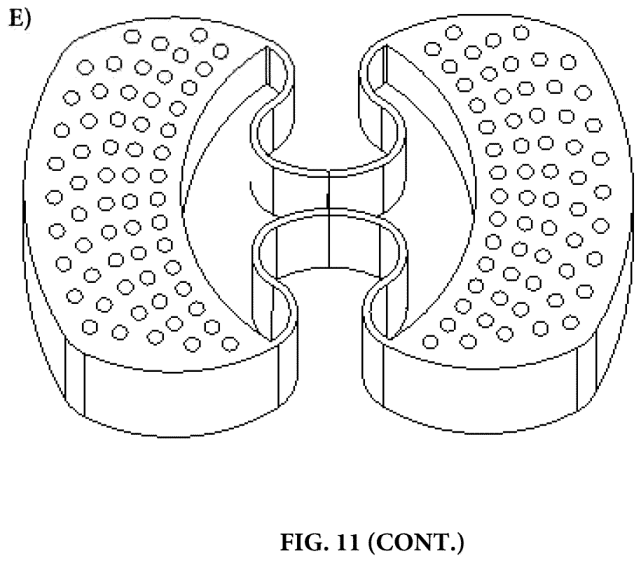

FIG. 11 shows illustrations of embodiments of the invention. Panel A shows a body, such as a body made of titanium, that is hollow to host biologics. Panel B shows a body composed by structures imitating bone trabeculae. Panel C shows a body with host inserts in other material such as PEEK. Panel D shows an illustration of function as inner frame for bodies made of other material such as PEEK. Panel E shows a body made with a porous structure or a of a porous material.

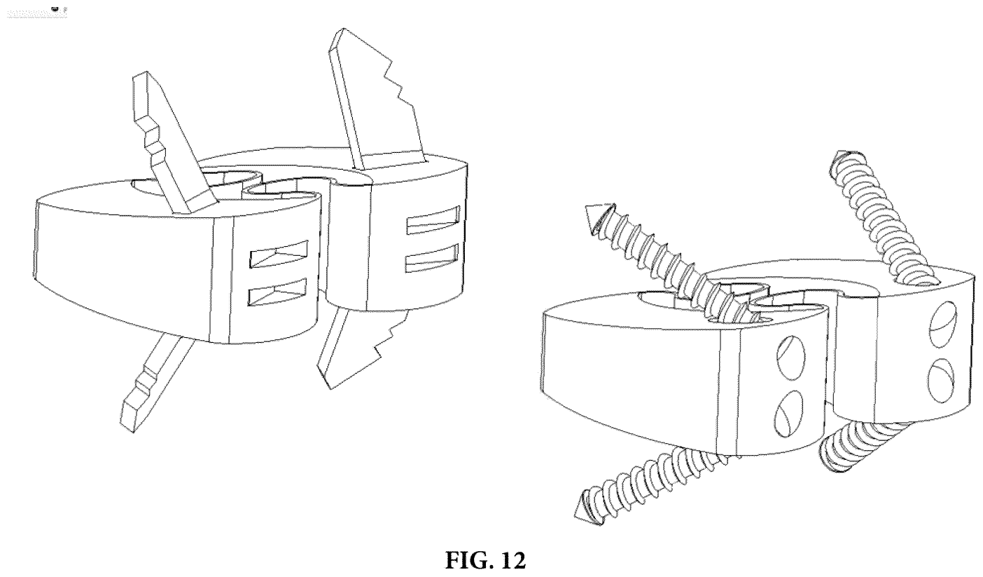

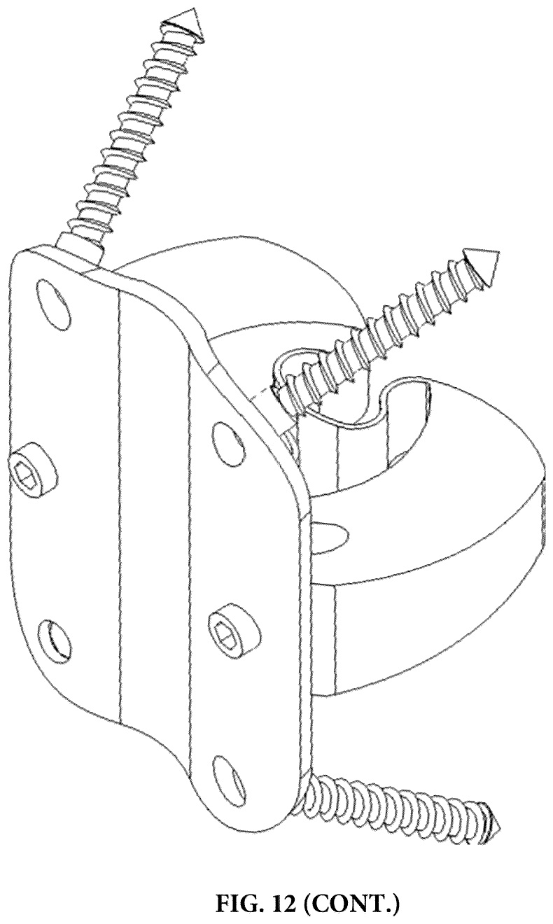

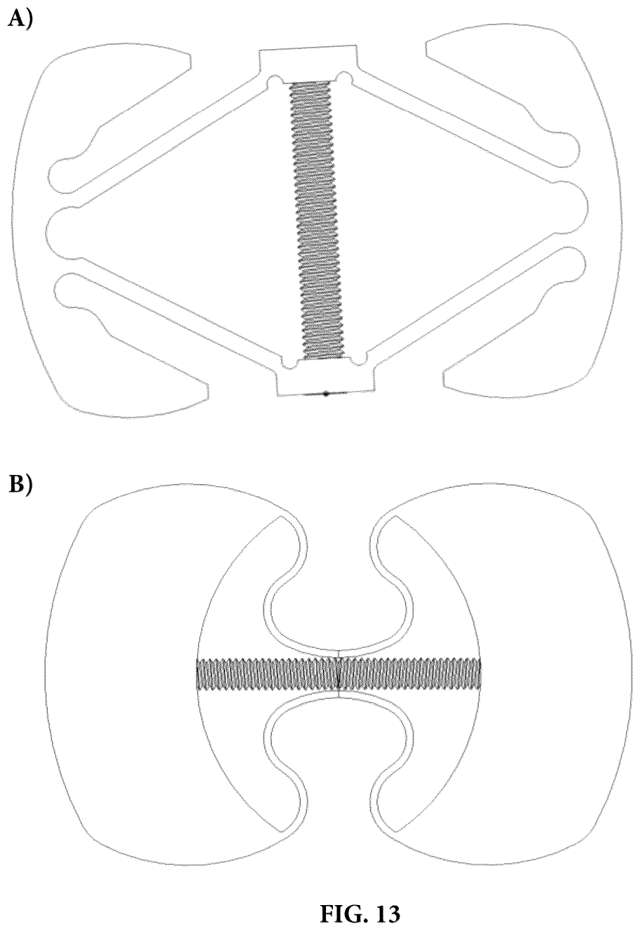

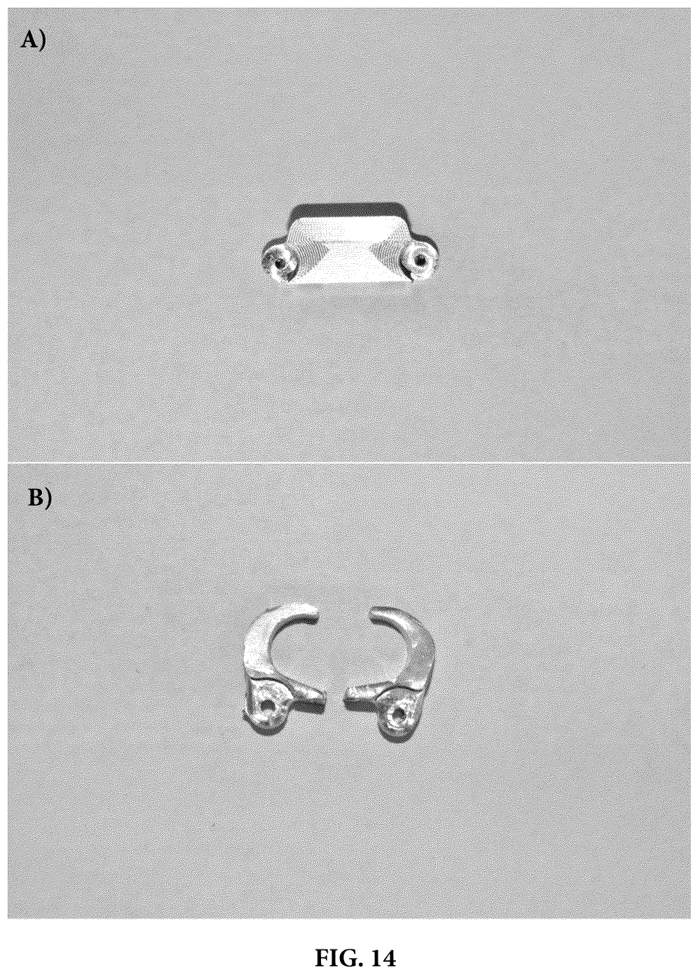

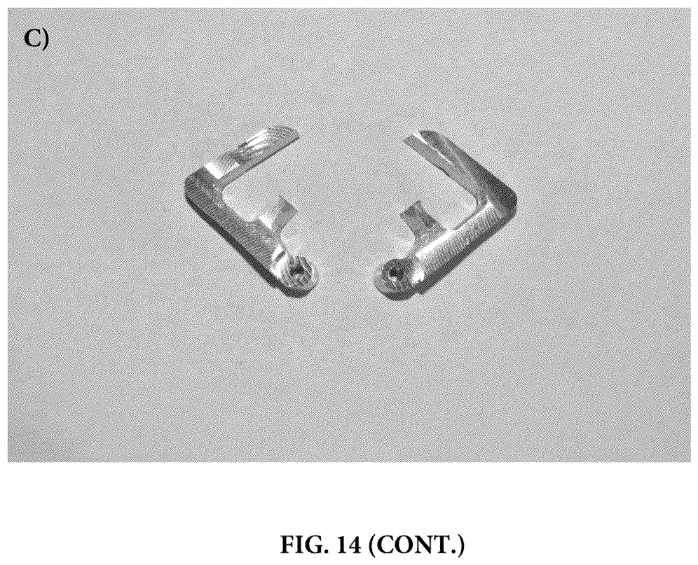

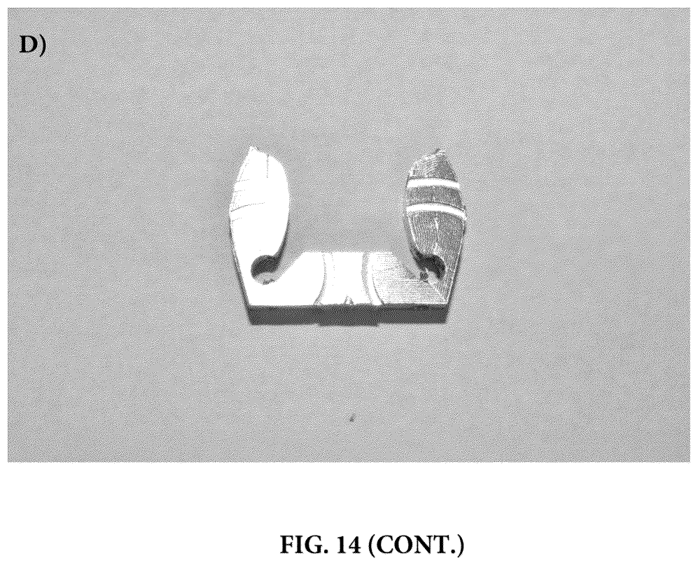

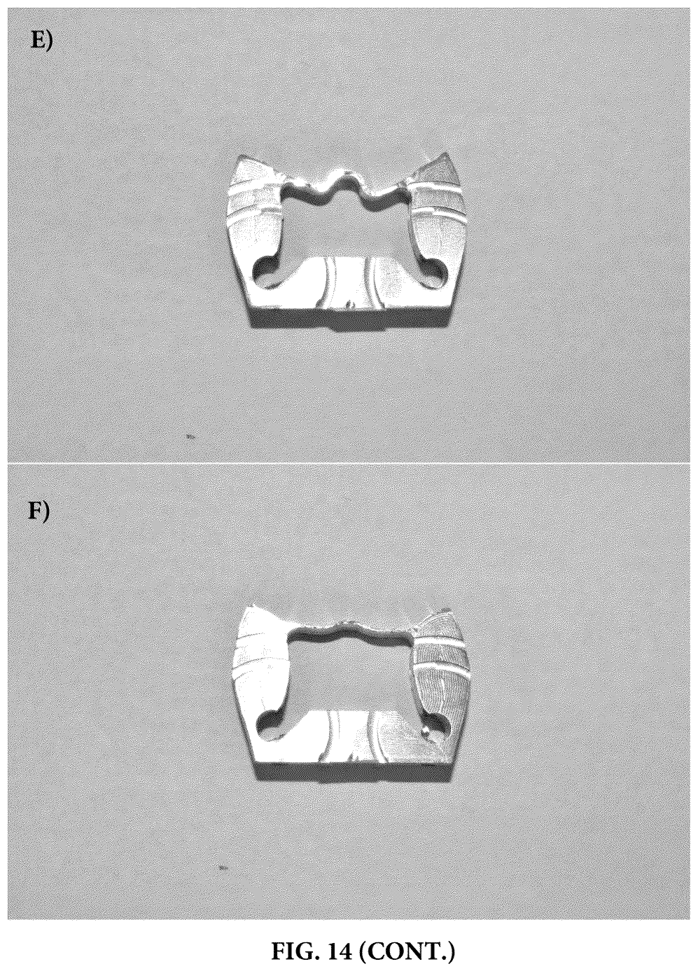

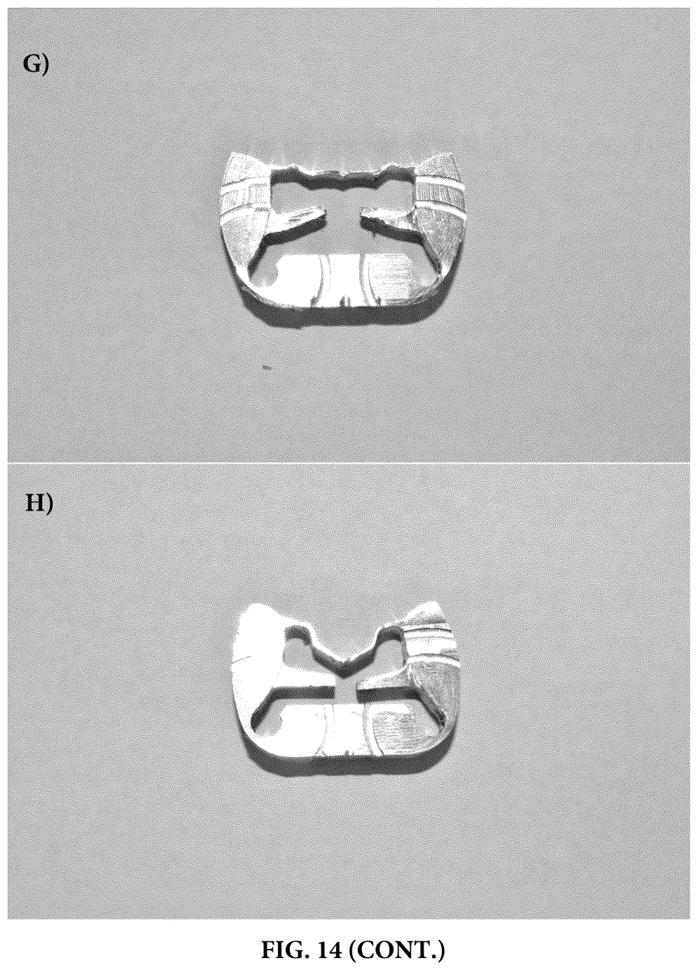

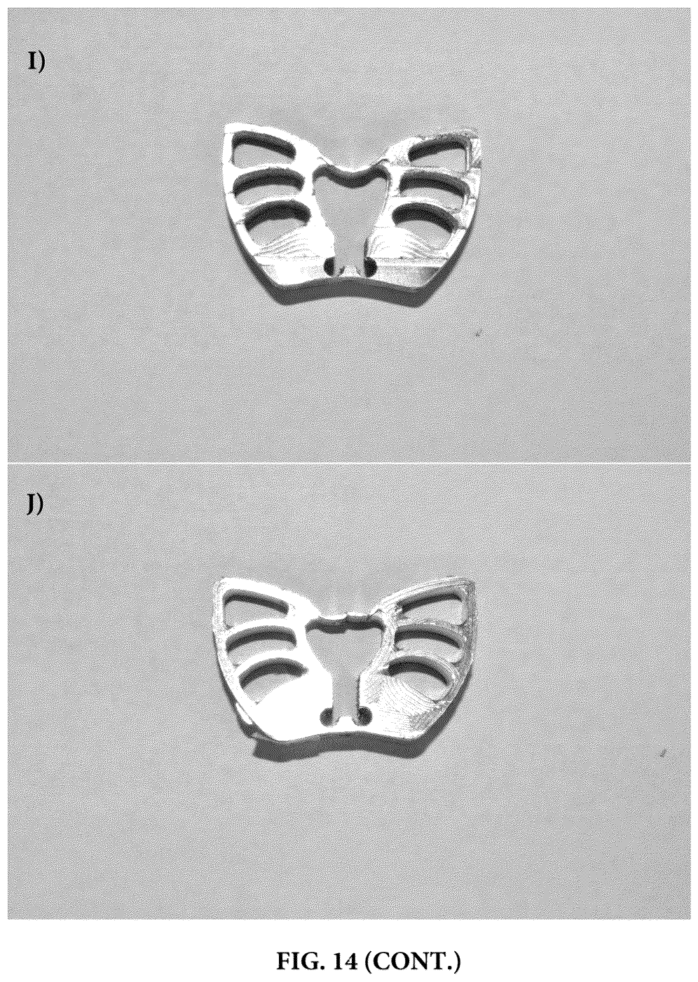

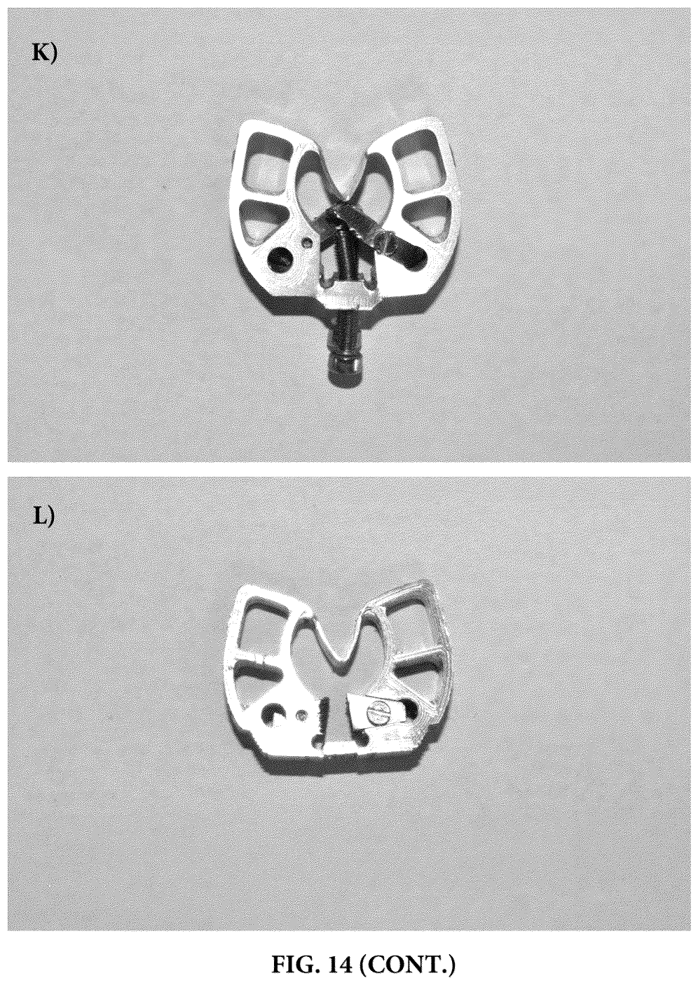

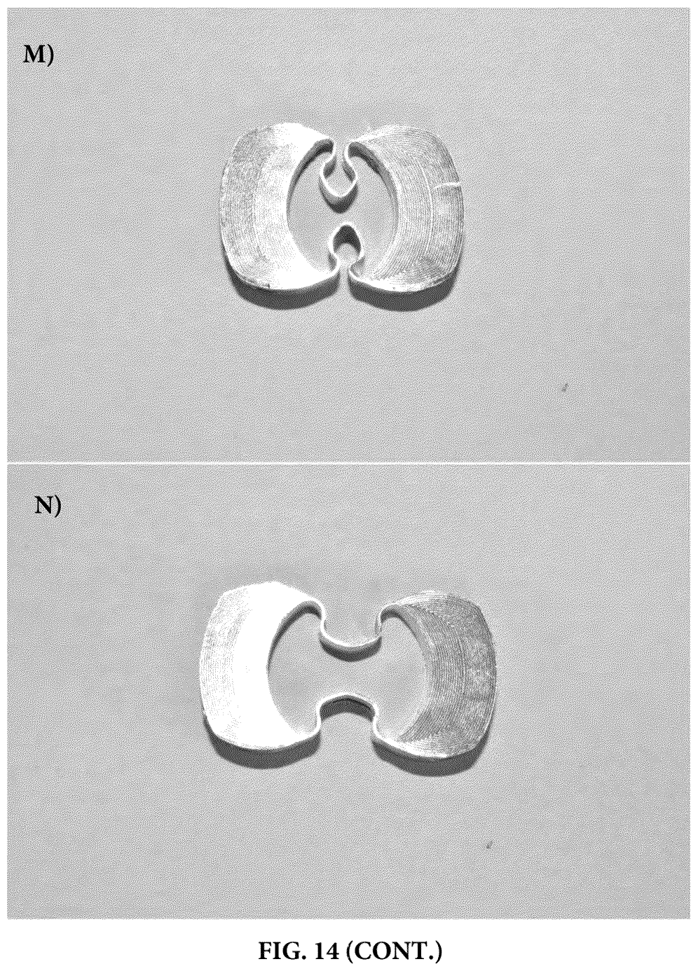

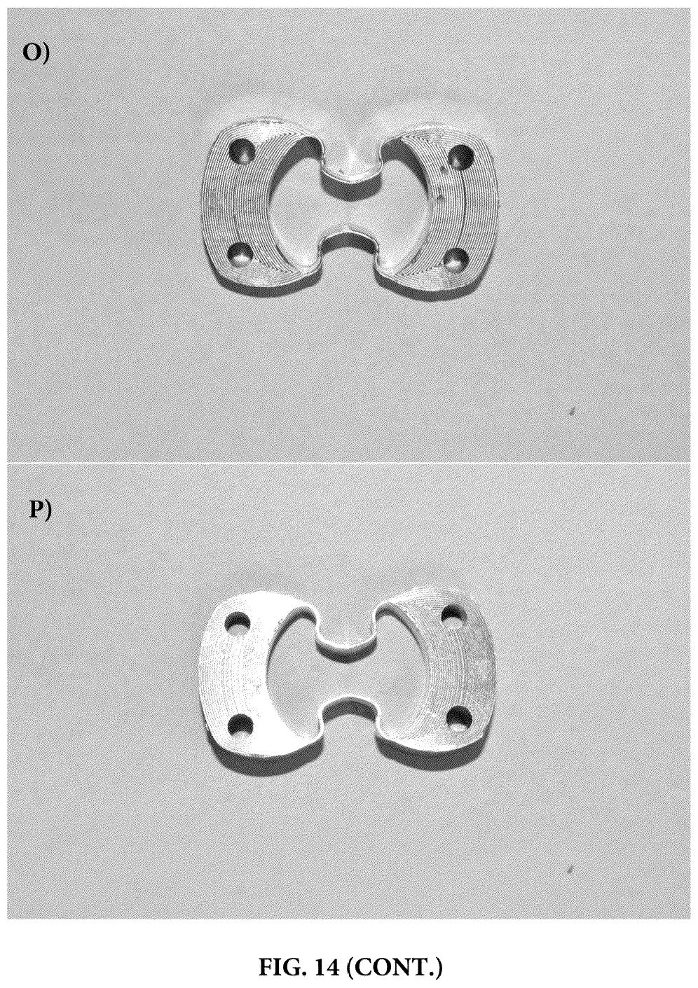

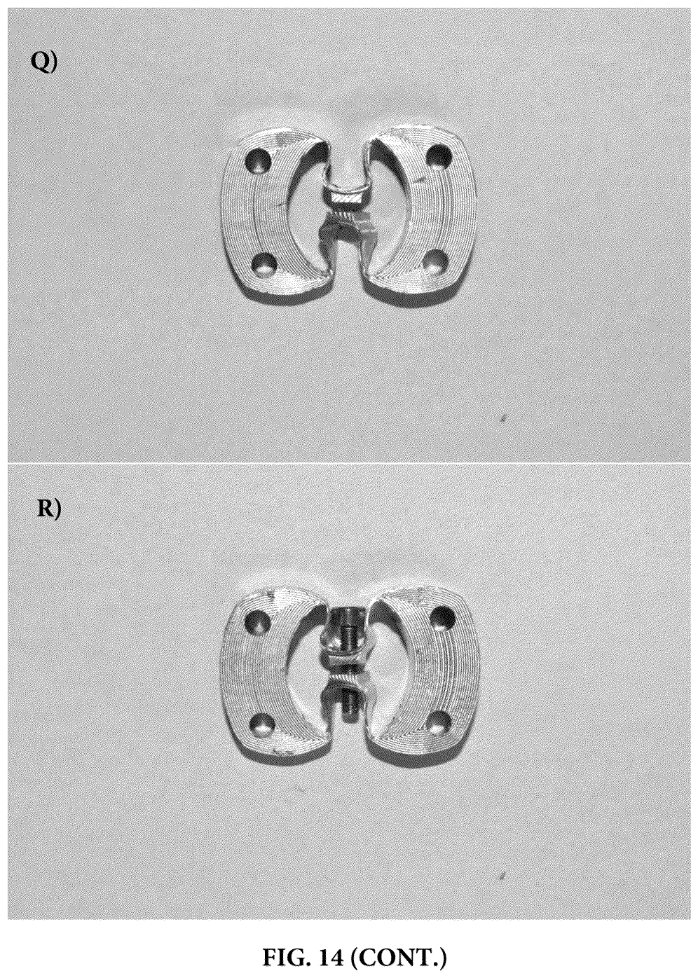

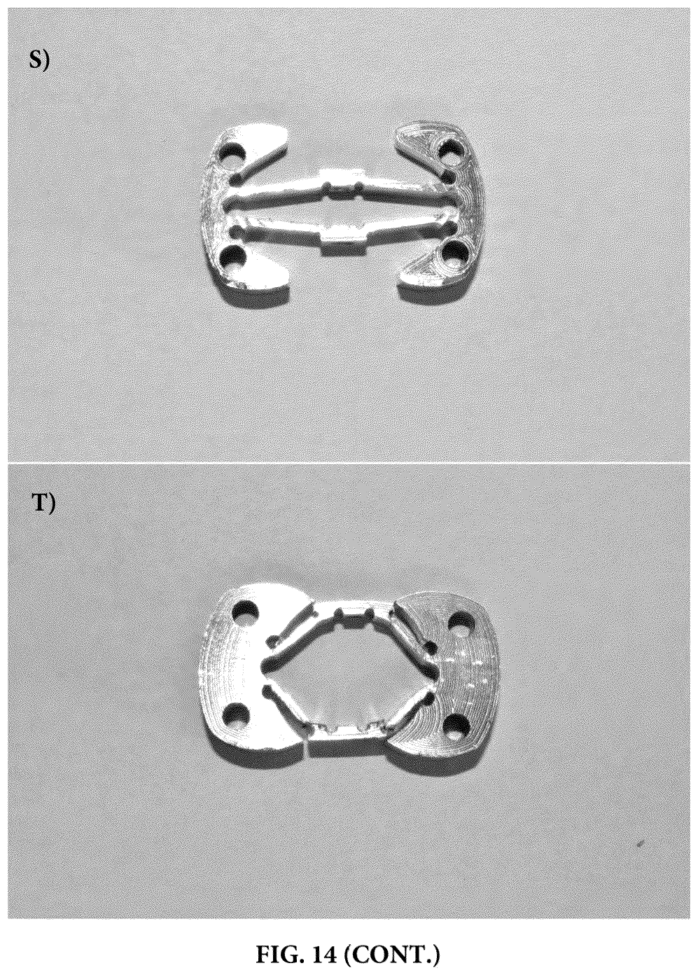

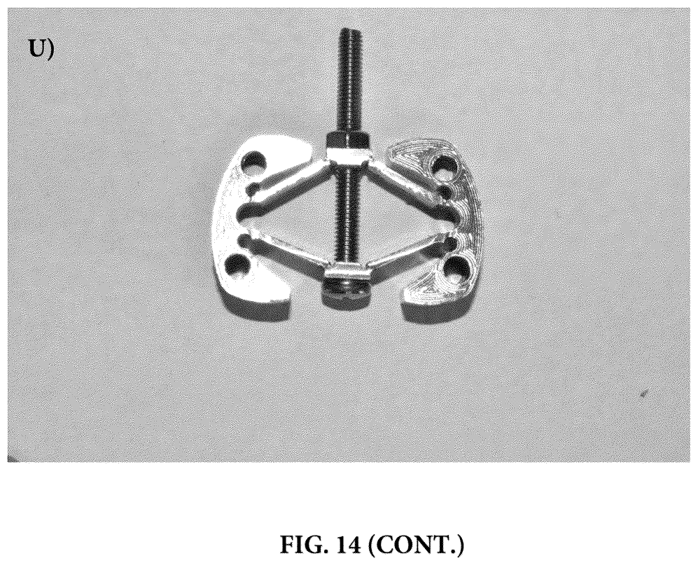

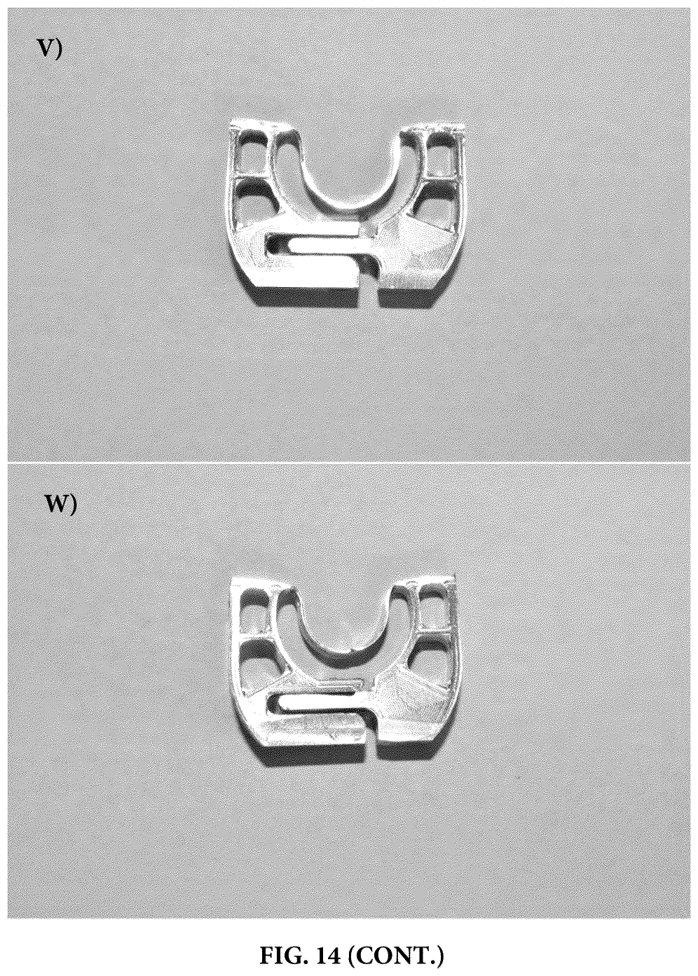

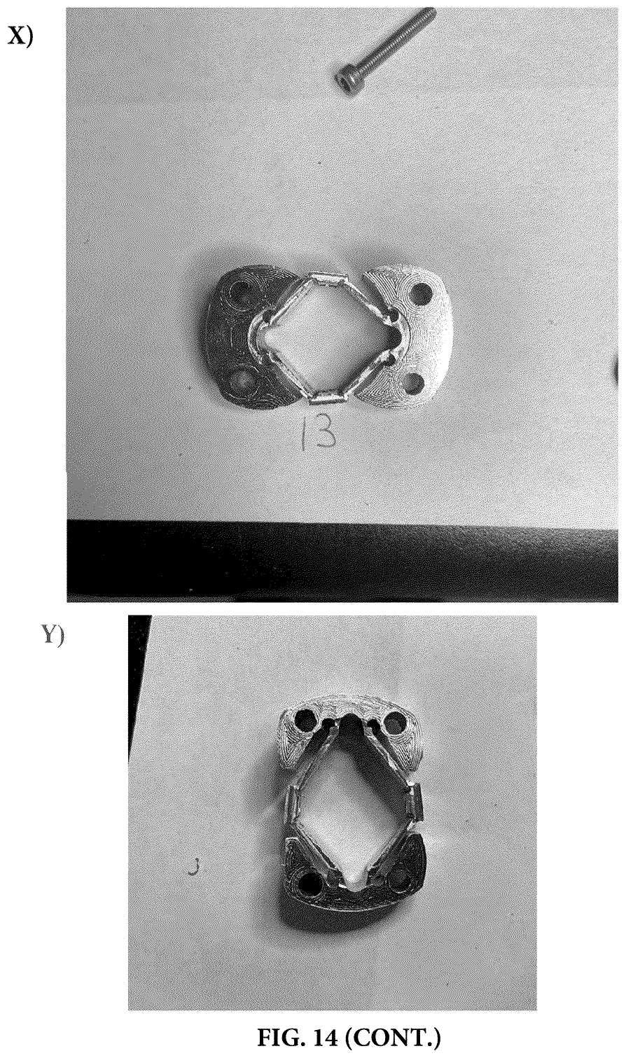

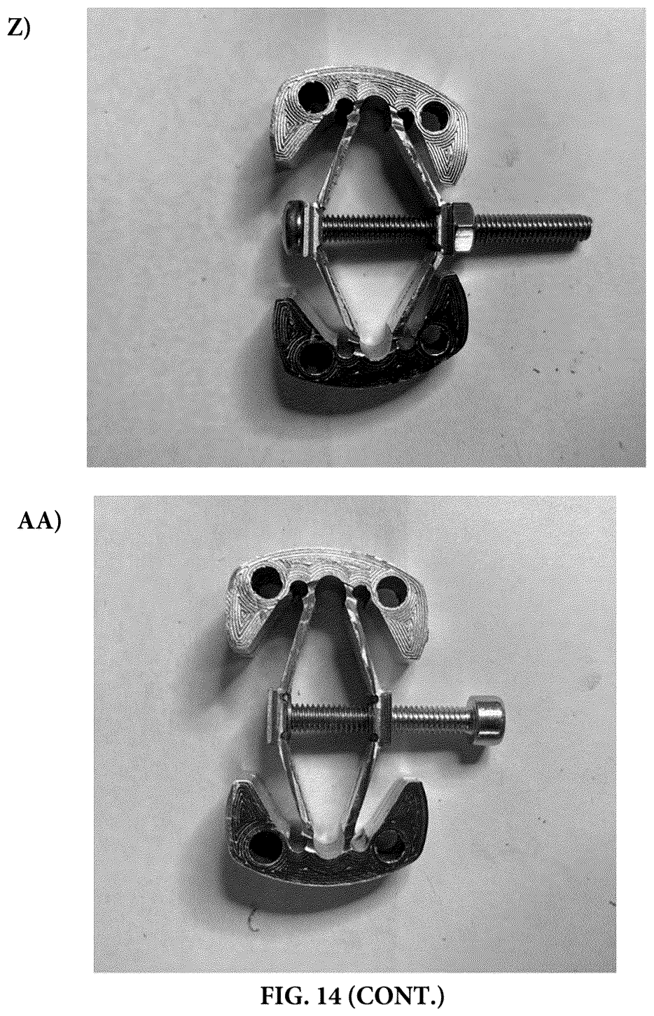

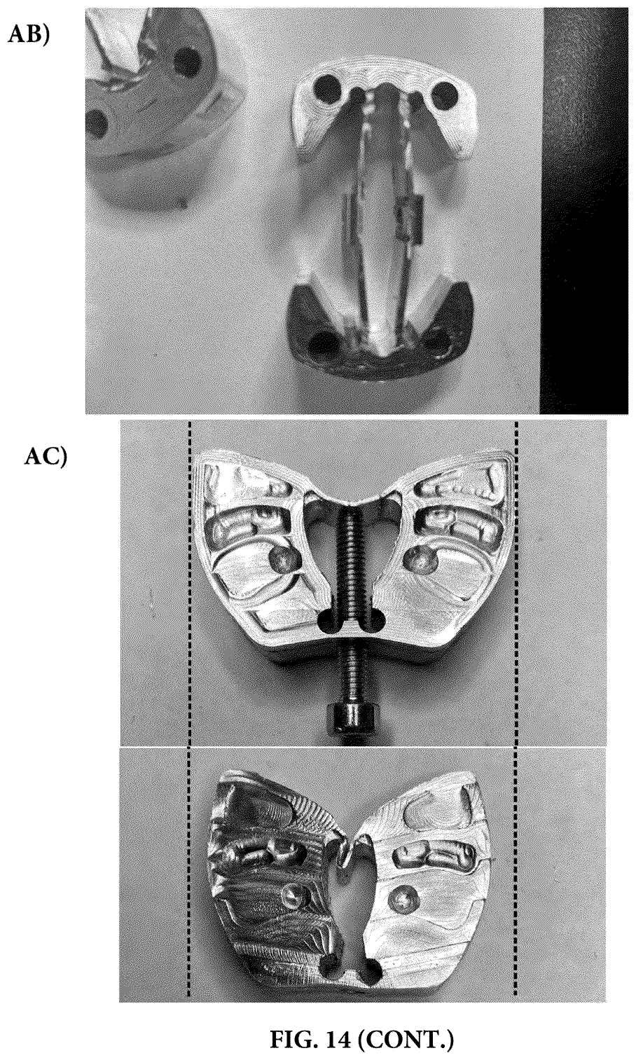

The invention in embodiments can also be provided of elements such as screws, blades, or plates, to easy the anchoring to the surrounding bones (see FIG. 12).

The expansion of the invention can be actuated or maintained through threaded elements disposed internally between the bodies (see FIG. 12, panel A) or between the linking elements (panel B).

The invention can be used for all the spine segments, and it can be indicated for the segments ranging from C2 to T1 and from L2 to S1.

The invention is configured to expand about 10 mm (e.g., expanding from a footprint of width 32 mm to a footprint of width 42 mm) and can have a cuneiform sagittal section with angle ranging from 0 to 30 degrees. It can host bone screw sizes in diameter ranging from 3.5 to 5.5 mm while blades can be of length ranging from 8 to 16 mm with thickness that varies from 1 mm to 2.5 mm.

The material hosted in the device at time of implantation can be of various nature and examples include, but are not limited to: Tricalcium phosphate, Bone morphogenetic proteins, demineralized bone matrix, stem cells, and Cortical or Cancellous Crushed bones.

### Example 2

### Evaluation on Bone Surrogates of the Influence of Epiphyseal Rim Contact on Construct Stiffness in Anterior Lumbar Interbody Fusion

### Introduction

Spinal fusion is a surgical treatment option for patients suffering with severe chronic back pain. Spinal fusion surgeries involve inserting an intervertebral body fusion device (cage) into the disk space to restore the disk height and further stabilize the motion segment with supplemental fixation to promote biological fusion. Main failure mode of ALIF is cage subsidence that has been documented in up to 70% of cases. Many authors have indicated that components must be selected maximizing endplate coverage but most of the implants are only available in few dimensional variations and shapes that do not match endplate shapes. Therefore, the selection oriented do maximize endplate coverage does not necessarily result in greater contact with the most rigid areas of the vertebra. Given the proven heterogeneity of the endplate in terms of stiffness and strength, in this study we aim to quantify the advantages in terms of construct stiffness for implants position that favor contact with the epiphyseal rim. Without wishing to be bound by theory, epiphyseal rim contact results in a significant increment of construct stiffness.

### Materials and Methods

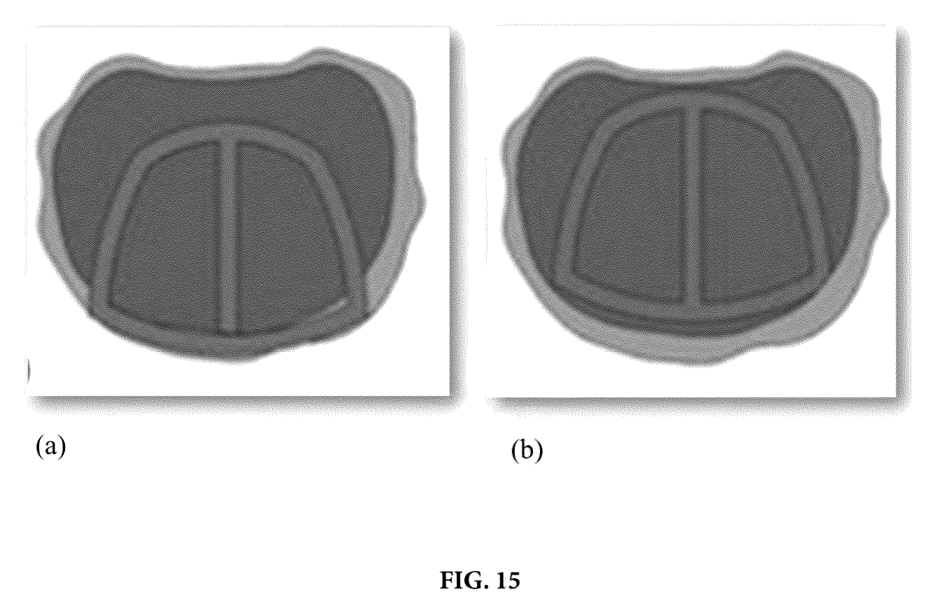

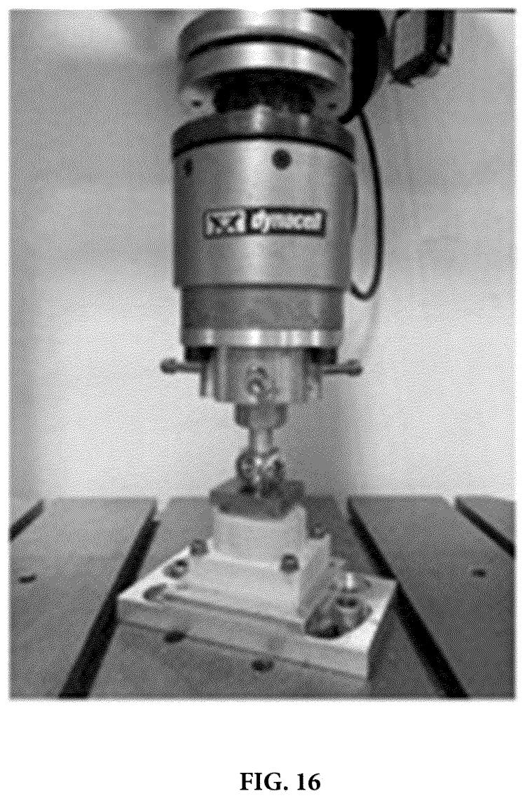

L4 vertebrae were reconstructed in Slicer 3d identifying cortical and trabecular regions from CT scans of cadaveric specimens. The surfaces were sectioned in correspondence of the superior endplate to extract profiles of the outer cortex and of the inner trabecular core. The profiles of the surrogates were obtained through linear extrusion of 20 mm in length and CNC machined in PCF 40 and PCF 15 to obtain cortical shell and trabecular core. An indented shape resembling an ALIF cage with a footprint of 36×30 mm was machined in stainless steel and positioned on the Instron mechanical testing machine with center of rotation in correspondence of the center of the endplate. The L4 bone surrogates were positioned in relation to the ALIF shaped plate to obtain, anterior and null overlap of the implant with the epiphyseal rim (FIG. 15). Mechanical testing (FIG. 16) was performed using an Instron Mechanical testing system with 5 mm/min of compressive displacement while recording force data at 100 Hz and every 1N increment. Construct stiffness was defined as the slope of the linear region of the force displacement curves. Variations were analyzed using Wilcoxon-Mann-Whitney at a significance level of 0.05.

### Non-Limiting, Exemplary Results

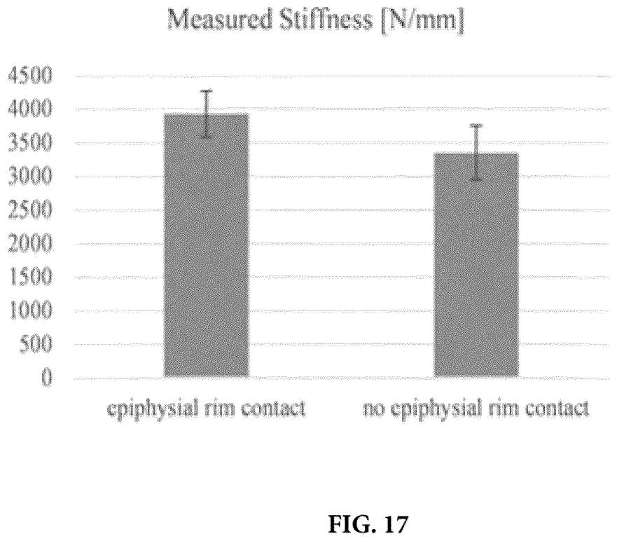

The stiffness among the two constructs did not show any difference up to values of 1000N (p=0.33). For higher loads, the construct with epiphyseal contact exhibited a stiffness of 3919 N/mm±348 higher than the value of 3340 N/mm±402 recorded for the configuration without epiphyseal rim contact (p=0.01) (FIG. 17).

### Non-Limiting Conclusions

In this study we used bone surrogates created using CT scans of cadaveric specimens. This approach allowed high reproducibility of our trials and ensured consistency of the configurations under investigation. Using a single ALIF cage profile varying only its position resulted in a variation of the construct stiffness. Our finding indicate that vertebra heterogeneity should be considered in ALIF planning.

### REFERENCES CITED IN THIS EXAMPLE

Briggs, H., Milligan, P. R., 1944. Chip fusion of the low back following exploration of the spinal canal. JBJS 26, 125-130.
Burkus, J. K., Gornet, M. F., Dickman, C. A., Zdeblick, T. A., 2002. Anterior lumbar interbody fusion using rhBMP-2 with tapered interbody cages. Clin. Spine Surg. 15, 337-349.
Cutler, Christopher B, et al. “Subsidence in a Novel 3D Printed Titanium ACDF Implant as Compared to PEEK.” ORS 2022 Annual Meeting Paper No. 2179, 2022.
Fogel G, Martin N, Williams G M, Unger J, Yee-Yanagishita C, Pelletier M, Walsh W, Peng Y, Jekir M. Choice of Spinal Interbody Fusion Cage Material and Design Influences Subsidence and Osseointegration Performance. World Neurosurg. 2022 March 26:S1878-8750(22)00380-1. doi: 10.1016/j.wneu.2022.03.087. Epub ahead of print. PMID: 35346883.
Fredricks, Joseph A., and David J. Nuckley. “Interbody Device Placement Is Critical to Subsidence Resistance.” ORS 2022 Annual Meeting Paper No. 1989, 2022.

### Example 3

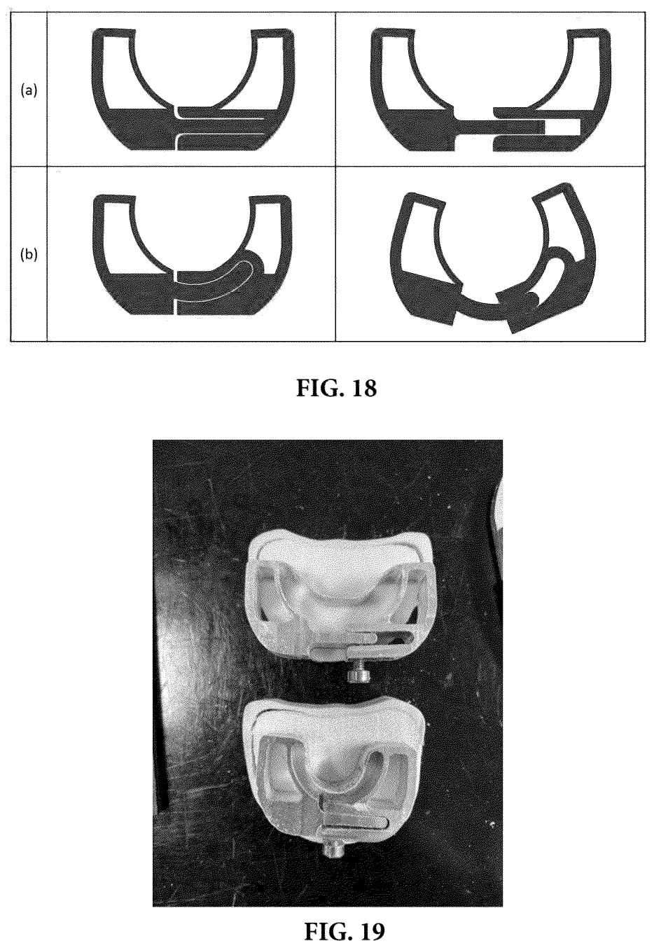

In the case of an implant made of two bodies able to be displaced to achieve the expansion, the relative displacement can be of various types. In the example shown in FIG. 18 the illustration shows the expansion linear or along a curved path.

Using a first prototype of the expandable cage we have performed experiments compressing the implant against bone surrogates of L4 vertebrae to determine the peak load to obtain a 2 mm subsidence. We have found that expanding the implant to match the width of the endplate results in a load of 3611±1139 N higher than the load of 3016±496 N recorded for the implant kept at its minimal width of 37 mm.

FIG. 19 shows an example of a prototype at its minimal width of 37 mm and expanded to match the vertebral width.

The obtainment of a conformed Interbody spacer can also be performed through CT scan or MRI images.

In the case of a conformable prebuilt spacer, the surgeon can evaluate the expansion needed on the CT images by positioning it in a 3D model of the adjacent vertebrae or assisted by an algorithm that interpolate the CT dataset in correspondence of the endplates to reconstruct their footprint.

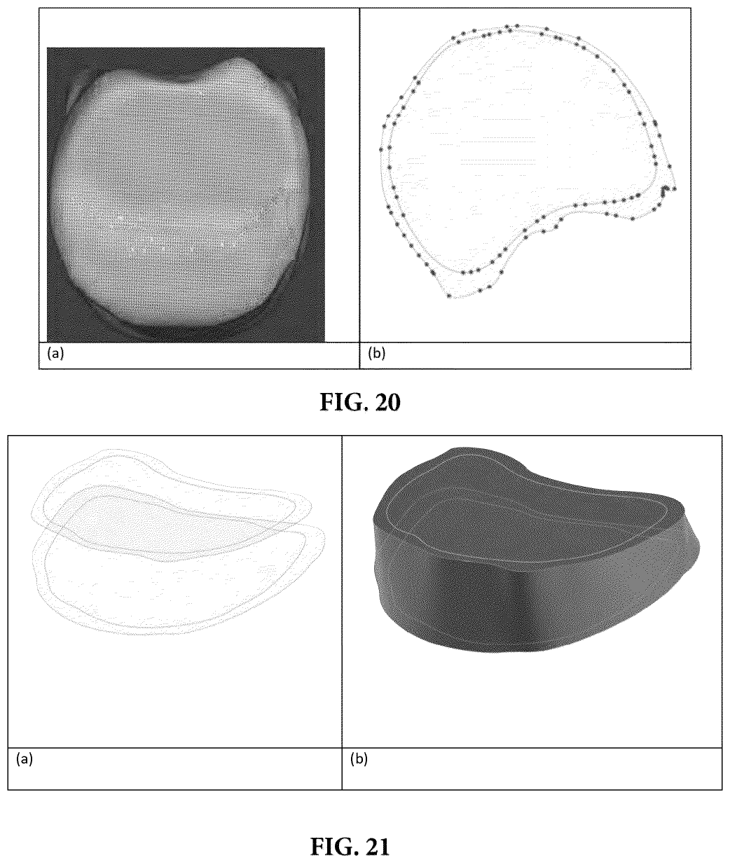

The user can then extract the footprint of the endplate or perform a cross section of the vertebra at any given depth to extract the thickness of the bone region with highest density therefore more able to bear load (see FIG. 20). FIG. 20 shows a CT reconstructed vertebra and its cross section at a given density threshold.

Similarly, the information on the endplate footprint can be used to shape an implant that is machined or 3D printed following one method similar to what is indicated in Saini et al. “Fussed filament fabrication-3D printing of poly-ether-ether-ketone (PEEK) spinal fusion cages”, Material Letters, Vol. 328, Article 133206. Extracted the endplates, the surgeons can decide a relative position between the endplates and the implant can be simply shaped trough the linear extrusion of the smaller endplate of the two. Alternatively, a loft surface can be created to blend the two endplates (see FIG. 21 panels a and b).

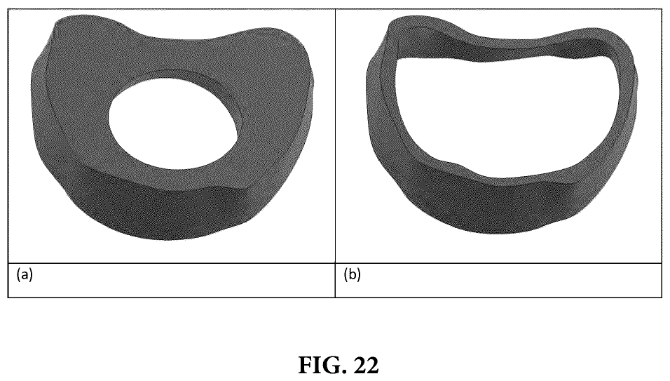

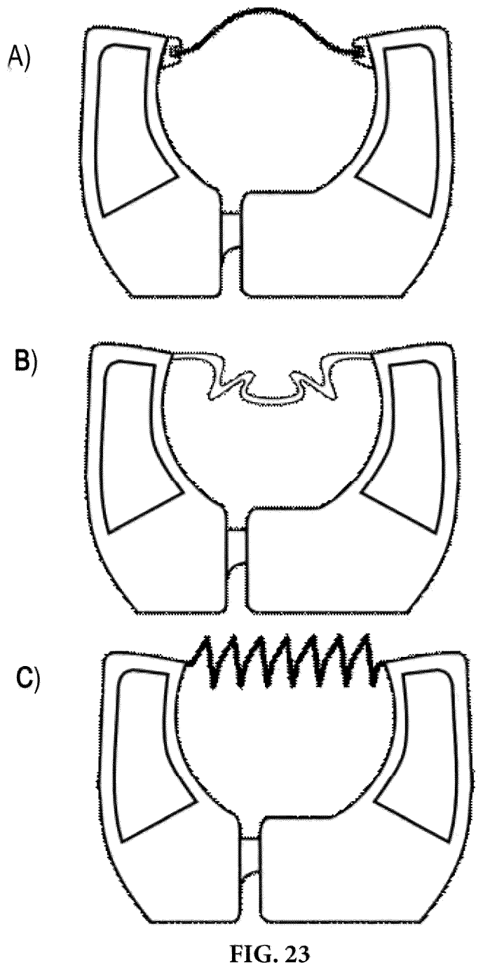

The implant can be internally shaped by a simple offset of the exterior surface so there is a void to host biologics. Alternatively, the inner shape of the implants is obtained through extraction of the cortical profile of the bone so the highest density in the implant is going to be positioned in correspondence of the epiphyseal rim (see FIG. 22).

FIG. 22 shows an illustration of an implant created through the interpolation of the cross-sections of two adjacent vertebrae here shown with a tubular void to host biologics (panel a) and also a void created interpolating the profiles of high-density bone seen in the cross-sections (panel b).

### Example 4

Disc degeneration is caused by many biochemical processes that occur with aging such as changes in permeability and water content (Guyer et al., 2023a). An estimated 266 million individuals worldwide are diagnosed with lumbar degenerative disc disease (DSD) annually (Ravindra et al., 2018). Anterior lumbar interbody fusion (ALIF) is used to relieve pain caused by degeneration of the intervertebral disc that is not amenable to conservative non-operative treatment (Mobbs et al., 2017; Rao et al., 2017a). Complications of the ALIF procedure include pseudoarthrosis (Wiesel & Albert, 2022), progressive cage subsidence (Parisien et al., 2022), foraminal stenosis (Guyer et al., 2023a), adjacent segment degeneration (Mobbs et al., 2017), metal allergy reaction, and ongoing pain (Wiesel & Albert, 2022). As a result, re-operations following complications have an incidence of 10-15% (Guyer et al., 2023). Subsidence depends on the patient's age, sex, bone mineral density, cage material, dimension, and placement (Igarashi et al., 2019; Patel et al., 2019a). Among the interbody fusion techniques, ALIF is the least likely surgical repair technique to subsidize when compared to lateral and posterior (Parisien et al., 2022). However, subsidence rates of anterior lumbar interbody fusion (ALIF) has between documented to vary between 6 and 24% (Parisien et al., 2022). While in some cases, subsidence has no significant impact on clinical outcomes in many cases can lead to adjacent disc deformity, decreased range of motion, foraminal stenosis, and resulting functional limitations in the patient's future (Rao et al., 2017). Cage sizing has been shown to influence subsidence load (Yuan et al., 2020) and is dependent on the surgical approach, the patient's anatomic characteristics such as lordosis angle, and the size of the vertebral body (Parisien et al., 2022; Patel et al., 2019). Other pre-operative factors to consider include the degree of disc degeneration, the presence of other abdominal pathology, and surgeon preference/experience (Lewandrowski et al., 2020; Patel et al., 2019). According to the current surgical technique (Wiesel & Albert, 2022), the surgeon's goal is to achieve the widest surface area contact possible without posing a risk to surrounding neurovascular structures or compromising the intervertebral foramina (Closkey et al., 1993; Mobbs et al., 2015a; Wiesel & Albert, 2022). It is well described that each vertebral end-plate is not homogenous in structure, which has a key effect on biomechanical resistance to subsidence (Wang et al., 2012). Furthermore, an in-vitro biomechanical experiment demonstrated that increased epiphyseal rim contact and a large vertebral body diameter were favorable toward resisting subsidence (Lowe et al., 2004b). However, for the lumbar spine, significant variance of vertebral body shape (Lowe et al., 2004a), endplate overall dimensions (Cantogrel et al., 2019), and ephysial rim depth have been documented while implants even if of various shapes are available in limited sizes and footprints (Chan et al., 2022; Igarashi et al., 2019; Patel et al., 2019a).

Therefore, the current study aims to identify the advantage of using endplate-tailored ALIF implants over standard-sized implants in determining ALIF subsidence load. In light of previous studies indicating the variability in shape and size of the epiphyseal rim, we hypothesize that the use of implants tailored to maximize ephysial rim coverage withstands higher subsidence loads than the standard implants.

CT scans of L5 vertebrae harvested from a total of 15 cadaveric spine spines with age of 80±7 yo (40% males, 60% females) were executed using a GE LightSpeed VCT scanner at a slice thickness of 0.625 mm. The vertebrae were reconstructed in a 3D slicer isolating endplates and cortical shell from the inner trabecular core. The obtained surfaces were imported in Fusion 360 (Autodesk, Mill Valley, California) and sectioned in correspondence of the superior endplate to extract profiles of the outer cortex and the inner trabecular core (Wang et al., 2011). For each reconstructed bone surrogate, for data analysis purpose, were extracted the total cortical area and the anterior cortical thickness in correspondence with the sagittal plane in the anteroposterior direction.

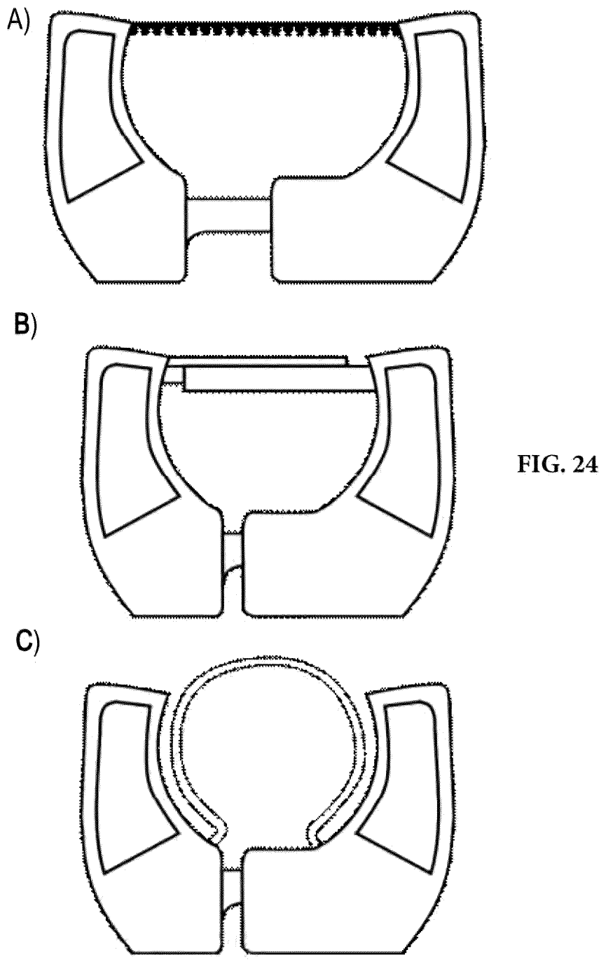

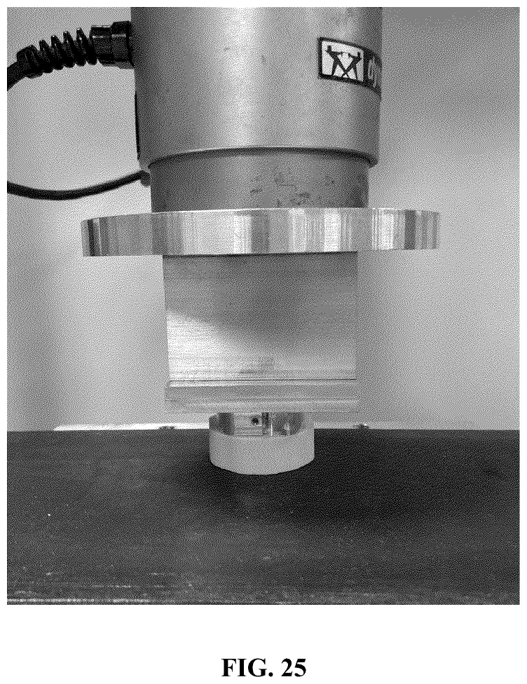

The profiles of the surrogates were obtained through the linear extrusion of 20 mm in length and CNC machined using bone surrogate foam in accordance with the ASTM F1839 standard. More specifically, the foam blocks were machined in PCF 40 and PCF 15 to obtain respectively cortical shell and trabecular core as adopted in previous studies (Fogel et al., 2022; Pinto et al., 2021). Following a previously used methodology (Fogel et al., 2022; Kiapour et al., 2023) the novel implant was positioned on the bone surrogates and compressed at a rate of 5 mm/min using an Instron 8874 (Instron, Norwood, MA), see FIG. 25. Each of the bone surrogates was tested with the implant in two configurations: unexpanded having a width of 37 mm (control) and expanded to maximize coverage (tailored). Data was acquired at 100 Hz and for every 1N increments from a reference preload of 5N. Mechanical characterization of the constructs was performed identifying peak load recorded within the 2 mm displacement used as reference position of subsidence as proposed for clinical classification of moderate subsidence in a previous study (Parisien et al., 2022), and stiffness evaluated as the angular coefficient of the linear regression of the obtained load-displacement curve. Normality of the data was evaluated on the data obtained for the control group using the Shapiro-Wilk test and differences between the two implant configurations were evaluated using paired T-test for means or Wilcoxon signed-rank test for non-parametric data, both at a significance level of 0.05. Proportionality of subsidence load and stiffness in relation to cortical surface area of the surrogates were analyzed using Pearson Correlation Coefficient. All the analysis were performed in R (LaZerte, 2016). In light of the experimental values found by Yuan at al. for interbody fusion cages tested in width increments of 5 mm, at a power of 80% we determined a minimal sample size needed of 4. A sample size of 15 was chosen for the study that resulted in 30 experiments.

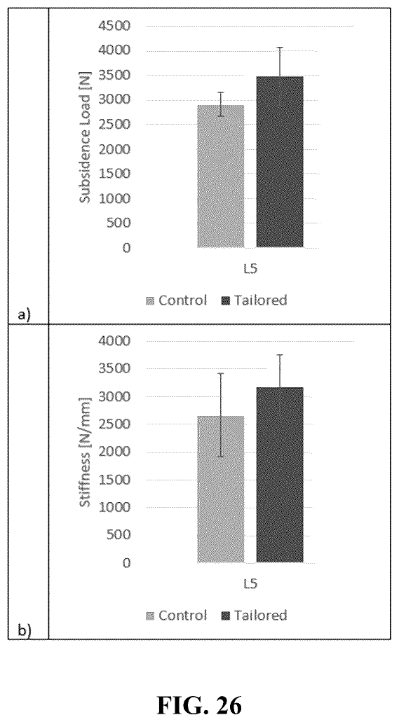

The control group resulted in a subsidence load of 2898N±243 that was normally distributed (p=0.428) and a construct stiffness of 2657 N/mm±749 also normally distributed (p=0.630). While stiffness was found normally distributed (p=0.179) the subsidence load was not found normally distributed for the tailored implants (p=0.003). Therefore, T-test was used for the stiffness value while Wilcoxon signed-rank test was used for the peak loads. The coefficient of determinations of the linear regressions used to calculate the construct stiffness were no different between the two groups (control r=0.96±0.28, tailored r=0.97±0.17, p=0.489). Tailored implants did not result in a significant improvement of stiffness (3165 N/mm±572, p=0.056, see FIG. 26). However, the subsidence load of 3478N±588 measured for the Tailored implants was significantly higher (p=0.002).

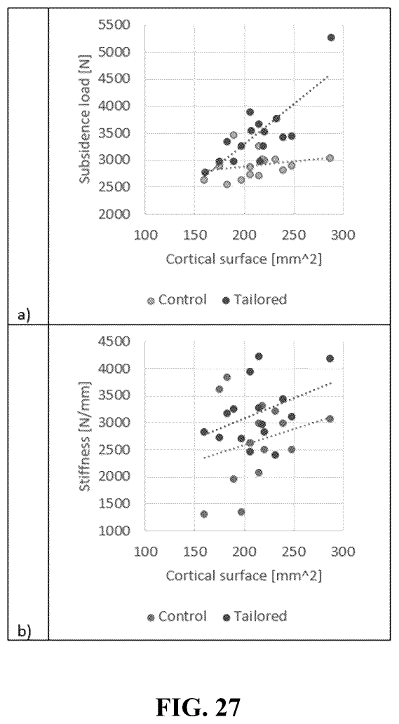

The subsidence load was not correlated to the surface of the cortical bone in the control group (r=0.260, p=0.350, y=0.034×+114.9) while was found to be correlated in the group with the tailored implants (r=0.796, p<0.001, y=0.043×+64.6, see FIG. 27A). In terms of construct stiffness, its value was notcorrelated to the surface of cortical bone (r=0.251, p=0.366, y=0.01×+184.4) in the control group and also in the tailored (r=0.411, p=0.127, y=0.02×+140.78, see FIG. 27B).

In spine interbody fusion vertebral heterogeneity plays an important factor in the resistance to subsidence (Palepu et al., 2019) and another element of challenge is given by the high variability in epiphyseal rim dimensions that has been documented to vary between 3 and 7 mm (Wang et al., 2012b).

The results of the current study provide experimental evidence that the use of an implant tailored to maximize epiphyseal coverage has the potential to increase the resistance to subsidence. Previous studies have suggested to seek wide contact area (Patel et al., 2019a) and it has been shown that 30% or above of endplate coverage is optimal for proper interbody fusion and improved load capacity (Closkey et al., 1993; Phan & Mobbs, 2016a). Even if it is common practice to bring a range of implant sizes to the operating room the variability in bone morphology is element of challenge in the implant selection (Hsieh et al., 2007; Patel et al., 2019a) because there is a limit to the amount of equipment that can be carried into the operating room (Choy et al., 2018). More recently, point of care manufacturing and 3D printing technologies are proposed as technological solution to improve outcomes in interbody fusion but there is a lack of evidence on surface modification of these devices. A case study in 2017 demonstrated the potential of 3d printed customized implants in reducing operation time due to ease of placement and avoidance of extensive complex reconstruction using bone grafts (Mobbs et al., 2017). However 3d printed implants require meticulous pre-operative planning, CT image elaboration and expensive equipment (Choy et al., 2018). For these reasons, the current study focused on the potential conferred by the adoption of an expandable implant.

Furthermore, we choose to focus on an expandable implant because ALIF surgery is performed with a retroperitoneal approach (Hsieh et al., 2007) and the use of an expandable implant is advantageous because allows for seeking a maximization of surface contact area (Buttermann et al., 2009; Mobbs et al., 2017), correct positioning in the center of the interspace between the vertebrae(Wiesel & Albert, 2022) while limiting tissues retraction typical of this surgery and source of complications. The main limitation of the experiments included in the current study is given by the use of bone surrogates in place of cadaveric specimens. This choice is common practice in orthopaedics to reduce variability in shape and density and experimental costs while improving the reproducibility of the experiments. Furthermore, we limited our study to specimen's representative of the lumbar anatomy only. This selection was imposed by the fact that the cadaveric spines used for the study were all harvested above the sacrum.

Another limitation is given by the fact that in the execution of the loading, we have only considered the compression but this was done considering the compression loading indicated by the ASTM F2077-22 standard (Standard Test Methods for Intervertebral Body Fusion Devices) and previous studies that have limited their analysis of interbody fusion constructs to the compression.

Furthermore, in spine interbody fusion there are many implant variations in terms material and supplemental fixation (Patel et al., 2019b). Titanium cage implants are durable and have the highest osteoconductive potential (Patel et al., 2019b; Phan & Mobbs, 2016b) while more recently Polyetheretherketone (PEEK) has gained popularity in light of shown lower subsidence rates and its radiolucent quality (Patel et al., 2019b). The current study focused on the shape variability that can be conferred to the implants independently from the material or the porosity conferred to host osteobiologics (BMP-2, DMP) often utilized to facilitate the speed of fusion post-operatively (Chan et al., 2022; Guyer et al., 2023a).

Another experimental piece of evidence we have found is that the instrumentation with the tailored implant resulted in a load proportional to the cross-section of cortical bone. The potential to reduce progressive subsidence post-operatively may lead to less morbidity and subsequent need for revision operations. The extension of this particular finding is limited by the simplifications adopted in the creation of the bone surrogates. While this needs to be corroborated with experiments on real vertebrae this opens to the potential of estimating preoperatively the subsidence load in addition to the other element commonly measured preoperatively such as foraminal height, local disc angle, and lumbar lordosis.

Increasing resistance to subsidence is paramount in lumbar interbody fusion. This study has shown the potential of tailored implants to improve this resistance. Furthermore, the potential relationship between cortical bone cross-section and subsidence warrants the exploration of predictive models that could be used in surgical planning.

### REFERENCES IN THIS EXAMPLE

Buttermann, G. R., Beaubien, B. P., Freeman, A. L., Stoll, J. E., & Chappuis, J. L. (2009). Interbody device endplate engagement effects on motion segment biomechanics. The Spine Journal, 9(7), 564-573. https://doi.org/10.1016/j.spinee.2009.03.014
Cantogrel, P., Schuller, S., Lefebvre, F., Charles, Y. P., & Steib, J.-P. (2019). Lower lumbar vertebra size and anatomic variation: An Anatomo-Radiologic Study. Orthopaedics & Traumatology, Surgery & Research: OTSR, 105(6), 1137-1141. https://doi.org/10.1016/j.otsr.2019.06.012
Chan, J. L., Bae, H. W., Farber, S. H., Uribe, J. S., Eastlack, R. K., & Walker, C. T. (2022). Evolution of Bioactive Implants in Lateral Interbody Fusion. International Journal of Spine Surgery, 16(S1), S61. https://doi.org/10.14444/8237
Choy, W. J., Parr, W. C. H., Phan, K., Walsh, W. R., & Mobbs, R. J. (2018). 3-dimensional printing for anterior cervical surgery: a review. Journal of Spine Surgery (Hong Kong), 4(4), 757-769. https://doi.org/10.21037/jss.2018.12.01
Closkey, R. F., Parsons, J. R., Lee, C. K., Blacksin, M. F., & Zimmermant, M. C. (1993). Mechanics of Interbody Spinal Fusion. Spine, 18(8), 1011-1015. https://doi.org/10.1097/00007632-199306150-00010
Fogel, G., Martin, N., Lynch, K., Pelletier, M. H., Wills, D., Wang, T., Walsh, W. R., Williams, G. M., Malik, J., Peng, Y., & Jekir, M. (2022). Subsidence and fusion performance of a 3D-printed porous interbody cage with stress-optimized body lattice and microporous endplates—a comprehensive mechanical and biological analysis. The Spine Journal: Official Journal of the North American Spine Society, 22(6), 1028-1037. https://doi.org/10.1016/j.spinee.2022.01.003
Guyer, R. D., Zigler, J. E., Blumenthal, S. L., Shellock, J. L., & Ohnmeiss, D. D. (2023a). Evaluation of Anterior Lumbar Interbody Fusion Performed Using a Stand-Alone, Integrated Fusion Cage. International Journal of Spine Surgery, 17(1), 1. https://doi.org/10.14444/8354
Guyer, R. D., Zigler, J. E., Blumenthal, S. L., Shellock, J. L., & Ohnmeiss, D. D. (2023b). Evaluation of Anterior Lumbar Interbody Fusion Performed Using a Stand-Alone, Integrated Fusion Cage. International Journal of Spine Surgery, 17(1), 1. https://doi.org/10.14444/8354
Hsieh, P. C., Koski, T. R., O'Shaughnessy, B. A., Sugrue, P., Salehi, S., Ondra, S., & Liu, J. C. (2007). Anterior lumbar interbody fusion in comparison with transforaminal lumbar interbody fusion: implications for the restoration of foraminal height, local disc angle, lumbar lordosis, and sagittal balance. Journal of Neurosurgery: Spine, 7(4), 379-386. https://doi.org/10.3171/SPI-07/10/379
Igarashi, H., Hoshino, M., Omori, K., Matsuzaki, H., Nemoto, Y., Tsuruta, T., & Yamasaki, K. (2019). Factors Influencing Interbody Cage Subsidence Following Anterior Cervical Discectomy and Fusion. Clinical Spine Surgery, 32(7). https://journals.lww.com/jspinaldisorders/Fulltext/2019/08000/Factors_Influencing_Interbod y_Cage_Subsidence.6.aspx
Kiapour, A., Massaad, E., Kodigudla, M. K., Kelkar, A., Begley, M. R., Goel, V. K., Block, J. E., & Shin, J. H. (2023). Resisting subsidence with a truss Implant: Application of the “Snowshoe” principle for interbody fusion devices. Journal of Biomechanics, 155, 111635. https://doi.org/10.1016/J.JBIOMECH.2023.111635
LaZerte, S. (2016, November 16). How to Cite R and R Packages. OpenSci.
Lewandrowski, K.-U., Ferrara, L., & Cheng, B. (2020). Expandable Interbody Fusion Cages: An Editorial on the Surgeon& #039; Perspective on Recent Technological Advances and Their Biomechanical Implications. International Journal of Spine Surgery, 14(s3), S56. https://doi.org/10.14444/7127
Lowe, T. G., Hashim, S., Wilson, L. A., O'Brien, M. F., Smith, D. A. B., Diekmann, M. J., & Trommeter, J. (2004a). A biomechanical study of regional endplate strength and cage morphology as it relates to structural interbody support. Spine, 29(21), 2389-2394. https://doi.org/10.1097/01.brs.0000143623.18098.e5
Lowe, T. G., Hashim, S., Wilson, L. A., O'Brien, M. F., Smith, D. A. B., Diekmann, M. J., & Trommeter, J. (2004b). A biomechanical study of regional endplate strength and cage morphology as it relates to structural interbody support. Spine, 29(21), 2389-2394. https://doi.org/10.1097/01.brs.0000143623.18098.e5
Mobbs, R. J., Coughlan, M., Thompson, R., Sutterlin, C. E., & Phan, K. (2017). The utility of 3D printing for surgical planning and patient-specific implant design for complex spinal pathologies: case report. Journal of Neurosurgery: Spine, 26(4), 513-518. https://doi.org/10.3171/2016.9.SPINE16371
Mobbs, R. J., Phan, K., Malham, G., Seex, K., & Rao, P. J. (2015a). Lumbar interbody fusion: techniques, indications and comparison of interbody fusion options including PLIF, TLIF, MI-TLIF, OLIF/ATP, LLIF and ALIF. Journal of Spine Surgery (Hong Kong), 1(1), 2-18. https://doi.org/10.3978/j.issn.2414-469X.2015.10.05
Mobbs, R. J., Phan, K., Malham, G., Seex, K., & Rao, P. J. (2015b). Lumbar interbody fusion: techniques, indications and comparison of interbody fusion options including PLIF, TLIF, MI-TLIF, OLIF/ATP, LLIF and ALIF. Journal of Spine Surgery (Hong Kong), 1(1), 2-18. https://doi.org/10.3978/j.issn.2414-469X.2015.10.05
Palepu, V., Helgeson, M. D., Molyneaux-Francis, M., & Nagaraja, S. (2019). The Effects of Bone Microstructure on Subsidence Risk for ALIF, LLIF, PLIF, and TLIF Spine Cages. Journal of Biomechanical Engineering, 141(3). https://doi.org/10.1115/1.4042181
Parisien, A., Wai, E. K., ElSayed, M. S. A., & Frei, H. (2022). Subsidence of Spinal Fusion Cages: A Systematic Review. International Journal of Spine Surgery, 16(6), 1103. https://doi.org/10.14444/8363
Patel, D. V., Yoo, J. S., Karmarkar, S. S., Lamoutte, E. H., & Singh, K. (2019a). Interbody options in lumbar fusion. Journal of Spine Surgery, 5(S1), S19-S24. https://doi.org/10.21037/jss.2019.04.04
Patel, D. V., Yoo, J. S., Karmarkar, S. S., Lamoutte, E. H., & Singh, K. (2019b). Interbody options in lumbar fusion. Journal of Spine Surgery, 5(S1), S19-S24. https://doi.org/10.21037/jss.2019.04.04
Phan, K., & Mobbs, R. J. (2016a). Evolution of Design of Interbody Cages for Anterior Lumbar Interbody Fusion. Orthopaedic Surgery, 8(3), 270-277. https://doi.org/10.1111/os.12259
Phan, K., & Mobbs, R. J. (2016b). Evolution of Design of Interbody Cages for Anterior Lumbar Interbody Fusion. Orthopaedic Surgery, 8(3), 270-277. https://doi.org/10.1111/os.12259
Pinto, O. T., Laforce, I. F., Badra, D. V., & de Mello Roesler, C. R. (2021). A New Test Method for In Vitro Evaluation of Pedicle Screw Loosening Potential. Journal of Medical Devices, 15(4). https://doi.org/10.1115/1.4052517
Rao, P. J., Phan, K., Giang, G., Maharaj, M. M., Phan, S., & Mobbs, R. J. (2017a). Subsidence following anterior lumbar interbody fusion (ALIF): a prospective study. Journal of Spine Surgery, 3(2), 168-175. https://doi.org/10.21037/jss.2017.05.03
Rao, P. J., Phan, K., Giang, G., Maharaj, M. M., Phan, S., & Mobbs, R. J. (2017b). Subsidence following anterior lumbar interbody fusion (ALIF): a prospective study. Journal of Spine Surgery, 3(2), 168-175. https://doi.org/10.21037/jss.2017.05.03
Ravindra, V. M., Senglaub, S. S., Rattani, A., Dewan, M. C., Hartl, R., Bisson, E., Park, K. B., & Shrime, M. G. (2018). Degenerative Lumbar Spine Disease: Estimating Global Incidence and Worldwide Volume. Global Spine Journal, 8(8), 784-794. https://doi.org/10.1177/2192568218770769
Wang, Y., Battid, M. C., Boyd, S. K., & Videman, T. (2011). The osseous endplates in lumbar vertebrae: Thickness, bone mineral density and their associations with age and disk degeneration. Bone, 48(4), 804-809. https://doi.org/10.1016/J.BONE.2010.12.005
Wang, Y., Battid, M. C., & Videman, T. (2012a). A morphological study of lumbar vertebral endplates: radiographic, visual and digital measurements. European Spine Journal: Official Publication of the European Spine Society, the European Spinal Deformity Society, and the European Section of the Cervical Spine Research Society, 21(11), 2316-2323. https://doi.org/10.1007/s00586-012-2415-8
Wang, Y., Battid, M. C., & Videman, T. (2012b). A morphological study of lumbar vertebral endplates: radiographic, visual and digital measurements. European Spine Journal: Official Publication of the European Spine Society, the European Spinal Deformity Society, and the European Section of the Cervical Spine Research Society, 21(11), 2316-2323. https://doi.org/10.1007/s00586-012-2415-8
Wiesel, S. W., & Albert, T. J. (2022). Operative Techniques in Orthopaedic Surgery, Chapter 16—Anterior Lumbar Interbody Fusion, Disc Replacement, and Corpectomy. Wolters Kluwer Health.
Yuan, W., Kaliya-Perumal, A.-K., Chou, S. M., & Oh, J. Y.-L. (2020). Does Lumbar Interbody Cage Size Influence Subsidence? A Biomechanical Study. Spine, 45(2), 88-95. https://doi.org/10.1097/BRS.0000000000003194

### ADDITIONAL REFERENCES

Lee, C. S., Hwang, C. J., Lee, D. H., Kim, Y. T., & Lee, H. S. (2011). Fusion rates of instrumented lumbar spinal arthrodesis according to surgical approach: a systematic review of randomized trials. Clinics in orthopedic surgery, 3(1), 39-47. https://doi.org/10.4055/cios.2011.3.1.39
Kornblum, M. B., Turner, A. W., Cornwall, G. B., Zatushevsky, M. A., & Phillips, F. M. (2013). Biomechanical evaluation of stand-alone lumbar polyether-ether-ketone interbody cage with integrated screws. The spine journal: official journal of the North American Spine Society, 13(1), 77-84. https://doi.org/10.1016/j.spinee.2012.11.013
Maccormick, A. P., & Sharma, H. (2020). Analysis of the Variables Affecting the Incidence, Location, and Severity of Cage Subsidence Following Anterior Cervical Discectomy and Fusion Operation. International journal of spine surgery, 14(6), 896-900. https://doi.org/10.14444/7137
Bocahut, N., Audureau, E., Poignard, A., Delambre, J., Queinnec, S., Flouzat Lachaniette, C. H., & Allain, J. (2018). Incidence and impact of implant subsidence after stand-alone lateral lumbar interbody fusion. Orthopaedics & traumatology, surgery & research: OTSR, 104(3), 405-410. https://doi.org/10.1016/j.otsr.2017.11.018

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

What is claimed:

1. A device comprising,
lateral bodies configured for contact with a vertebral endplate of a subject, wherein the lateral bodies comprise a first lateral body and a second lateral body, wherein the first lateral body and the second lateral body are connected using linking elements, wherein the linking elements comprise a first linking element and a second linking element;
wherein the linking elements are configured for adjustable spacing of the lateral bodies relative to each other.

2. The device of claim 1, wherein the adjustable spacing comprises deformation of at least one of the first linking element and the second linking element.

3. The device of claim 2, wherein the deformation comprises expansion or contraction of the at least one of the first linking element and the second linking element.

4. The device of claim 3, wherein the first linking element connects corresponding posterior portions of the lateral bodies.

5. The device of claim 4, wherein expansion of the first linking element comprises expanding distance between the corresponding posterior portions.

6. The device of claim 5, wherein compression of the first linking element comprises contraction of distance between the corresponding posterior portions.

7. The device of claim 2, wherein the second linking element connects corresponding anterior portions of the lateral bodies.

8. The device of claim 7, wherein expansion of the second linking element comprises expanding distance between the corresponding anterior portions.

9. The device of claim 8, wherein compression of the second linking element comprises contraction of distance between the corresponding anterior portions.

10. The device of claim 9, wherein the lateral bodies and the linking elements form an interior space.

11. The device of claim 10, wherein at least one of the first and the second lateral body comprises a lateral body interior space.

12. A device comprising,
lateral bodies configured for contact with a vertebral endplate of a subject, wherein the lateral bodies comprise a first lateral body and a second lateral body, wherein the first lateral body and the second lateral body are connected using linking elements, wherein the linking elements comprise a first linking element and a second linking coupling;
wherein the linking elements are configured for adjustable spacing of the lateral bodies relative to each other.

13. The device of claim 12, wherein the first linking element connects corresponding posterior portions of the first lateral body and the second lateral body, wherein the first linking element is deformable.

14. The device of claim 13, wherein the linking coupling connects corresponding anterior portions of the second lateral body.

15. The device of claim 14, wherein the linking coupling comprises a sliding component integrally formed with and extending from the first lateral body.

16. The device of claim 15, wherein the linking coupling comprises a receiving chamber formed within the second lateral body, wherein the receiving chamber comprises an enclosed distal end and a proximal opening.

17. The device of claim 16, wherein movement of the sliding component in a direction of the closed distal end of the receiving chamber corresponds to compression of the first linking element and contraction of distance between the lateral bodies.

18. The device of claim 17, wherein movement of the sliding component in a direction away from the closed distal end of the receiving chamber corresponds to expansion of the first linking element and expansion of distance between the lateral bodies.

19. The device of claim 18, wherein the sliding component and the receiving chamber are linear shaped and the movement is linear.

20. The device of claim 19, wherein the sliding component and the receiving chamber are arcuate shaped and the movement is angular.

21. The device of claim 20, wherein a threaded component locks the sliding component in position within the receiving chamber.

22. A device comprising,
a first component comprising an anterior facing surface and a posterior facing surface;
a first claw and a second claw rotatably attached to a joint at opposing peripheral ends of the posterior facing surface, wherein each claw is independently rotatable around the respective joint, wherein the independently adjustable claws are configurable to increase the device's surface area coverage of an epiphyseal rim of a vertebral endplate.
